(19) United States
(12) Patent Application Publication
(10) Pub. No.: US 2026/0100271 A1
(43) Pub. Date: Apr. 9, 2026

(54) AUTOMATED DATA AGGREGATION WITH FILE ANALYSIS AND PREDICTIVE MODELING

(71) Applicant: Prefcards LLC, Las Vegas, NV (US)

(72) Inventors: Paul Reynolds, Las Vegas, NV (US); Brandon Reynolds, Las Vegas, NV (US); Neil McGuire, Broadway, NC (US); Chad Ramos, Las Vegas, NV (US)

(73) Assignee: Prefcards LLC, Las Vegas, NV (US)

(21) Appl. No.: 19/350,819

(22) Filed: Oct. 6, 2025

Related U.S. Application Data
(63) Continuation-in-part of application No. 19/212,510, filed on May 19, 2025, now abandoned.
Application No. 19/212,510 is a continuation of application No. 17/694,504, filed on Mar. 14, 2022, now Pat. No. 12,308,115.
(60) Provisional application No. 63/160,400, filed on Mar. 12, 2021.
(60) Provisional application No. 63/254,012, filed on Oct. 8, 2021.

(51) Int. Cl. G16H 40/20; G06F 3/0482; G06F 40/134; G06F 40/166; G06V 30/14; G06V 30/19

(52) U.S. Cl. CPC G16H 40/20; G06F 3/0482; G06F 40/134; G06F 40/166; G06V 30/1448; G06V 30/19173

(57) ABSTRACT

Systems, methods, and devices for data ingestion and aggregation, file analysis, and predictive modeling. A method includes generating an aggregated data form comprising surgical preference data, wherein the aggregated data form identifies a plurality of medical items, and wherein the aggregated data form is associated with a surgeon, a facility, and a surgery type. The method includes electronically communicating with an inventory management solution associated with the facility to retrieve inventory data for the plurality of medical items. The method includes receiving from a machine learning algorithm an amendment suggestion for the aggregated data form, wherein the amendment suggestion comprises one or more of: an amendment to a quantity of a first medical item, an identity of a first product satisfying the first medical item, or an addition of a second medical item not included in the aggregated data form.

### CROSS-REFERENCE TO RELATED APPLICATIONS

The disclosure is a continuation-in-part of U.S. patent application Ser. No. 19/212,510, filed May 19, 2025, entitled “AUTOMATED DATA AGGREGATION WITH FILE ANALYSIS AND PREDICTIVE MODELING,” which is a continuation of U.S. application Ser. No. 17/694,504 (now U.S. Pat. No. 12,308,115), filed Mar. 14, 2022, entitled “AUTOMATED DATA AGGREGATION WITH FILE ANALYSIS AND PREDICTIVE MODELING,” which claims the benefit of U.S. Provisional Ser. No. 63/160,400 , filed Mar. 12, 2021, entitled “PREDICTIVE MODELING FOR GENERATING AND MAINTAINING PREFERENCE CARDS ACROSS INDEPENDENT SYSTEMS” and claims the benefit of U.S. Provisional Ser. No. 63/254,012 , filed Oct. 8, 2021, entitled “PREDICTIVE MODELING FOR GENERATING AND MAINTAINING PREFERENCE CARDS ACROSS INDEPENDENT SYSTEMS.” The aforementioned patent applications are incorporated herein by reference in their entireties, including but not limited to those portions that specifically appear hereinafter, the incorporation by reference being made with the following exception: In the event that any portion of the aforementioned patent applications are inconsistent with this application, this application supersedes the aforementioned patent applications.

### TECHNICAL FIELD

The disclosure relates generally to data aggregation and particularly to data aggregation that leverages file analysis and predictive modeling.

### BACKGROUND

Numerous industries rely on consolidated, accurate, and easy-to-understand data. In some cases, this consolidated data may include, for example, a listing of tasks that must be performed, a listing of items to be collected, a recipe to be carried out, and so forth. It can be imperative that the consolidated data is accurate, up-to-date, and easy to maintain when tasks, items, and processes change over time.

Specifically, the healthcare field relies on strict adherence to protocol for successful operation. Healthcare procedures can be particularly complex because they combine efforts of numerous healthcare practitioners within a healthcare facility, and further include the use of pharmaceuticals, medical devices, and other items. Many practitioners and facilities implement the use of “preference cards,” or “doctor preference cards” (DPC) when performing a healthcare procedure. Preference cards are used in most operating rooms across hospitals, clinics, and surgical centers. Preference cards function like a recipe card and list the necessary equipment, instruments, and supplies needed for a successful procedure. Preference cards may include specific notes or comments that are meaningful to practitioners and other clinicians to provide the best care. It is important to know exactly which supplies need to be present in the operating room, and when to have those supplies available, to ensure a safe procedure and efficient and accurate billing of the procedure.

Traditional preference cards are handwritten by practitioners, and then a hardcopy of the preference card is stored at each facility where the practitioner performs procedures. Each practitioner may have numerous preference cards, including an independent preference card for each procedure the practitioner performs. Practitioners may additionally have different preference cards at different facilities to reflect the different inventory available at each facility. Practitioners cannot easily amend preference cards, and there is no system that permits practitioners to amend one preference card and then propagate those amendments to other facilities and procedures. Additionally, there is no efficient means for practitioners to share preference cards for certain procedures or provide advice on different items that can be used for a successful procedure.

Additionally, one large facility, such as a hospital or large surgical center, may store thousands of independent preference cards, wherein each preference card is associated with one practitioner and one procedure performed by that practitioner. These facilities cannot easily streamline their preference card system or move to a paperless system because this migration requires manually converting handwritten preference cards to a digital format. This analog-to-digital migration is cost prohibitive because it requires significant time and manpower. Therefore, it is desirable to introduce a streamlined means of ingesting, aggregating, and analyzing data, and then propagating the analyzed data into a file format that is easy to understand and manipulate.

Considering the foregoing, disclosed herein are systems, methods, and devices for data ingestion, unstructured file analysis, and generating preference card files that can easily be updated, synced, and propagated to other systems.

### BRIEF DESCRIPTION OF THE DRAWINGS

Non-limiting and non-exhaustive implementations of the present disclosure are described with reference to the following figures, wherein like reference numerals refer to like parts throughout the various views unless otherwise specified. Advantages of the present disclosure will become better understood with regard to the following description and accompanying drawings where:

FIG. 1A is a schematic diagram of a system for predictive modeling, file analysis, and management of aggregated data forms;

FIG. 1B is a schematic block diagram illustrating components of a data aggregation platform;

FIG. 2A is a schematic diagram of a system for storing data in a fault-tolerant manner across geographic locations;

FIG. 2B is a schematic diagram of a system for securely connecting a user with a data aggregation platform based on unique credentials;

FIG. 3 is a schematic diagram of a system for data communication between a data aggregation server and internal and external data sources;

FIG. 4 is a schematic diagram of a system for performing electronic data security measures on data received from an external data source;

FIG. 5A is a schematic block diagram of a system and method for applying a master aggregated data form to one or more facilities;

FIG. 5B is a schematic block diagram of a process flow for generating and suggesting procedure-specific and/or facility specific aggregated data form items based on a master aggregated data form and additional data sources;

FIG. 6 is a schematic block diagram of a process flow for scanning a scannable code to automatically redirect to a complete version of clean version of an aggregated data form;

FIG. 7 is a schematic block diagram of a dataflow for training an AI/ML engine, providing input data to the AI/ML engine, and receiving an output calculation from the AI/ML engine;

FIG. 8 is a screenshot of an example user interface for entering item selections for an aggregated data form;

FIG. 9 is a screenshot of an example user interface for quickly adding item selections to an aggregated data form;

FIG. 10 is a screenshot of an example user interface for building an aggregated data form;

FIG. 11 is a screenshot of an example user interface for calculating predicted procedure costs based on items selected on an aggregated data form;

FIG. 12 is a screenshot of an example user interface for providing cost-variation data for a certain procedure;

FIG. 13 is a screenshot of an example user interface for selecting, printing, viewing, and communicating aggregated data forms;

FIG. 14 is a screenshot of an applicable user interface and virtual file data-output by the data aggregation server;

FIG. 15 is a screenshot of an example user interface of the data aggregation platform;

FIG. 16 is a screenshot of an example user interface of the data aggregation platform;

FIG. 17A is a screenshot of a portion of an example user interface for presenting a virtual file output for an aggregated data form, wherein the portion illustrates the type of surgery, significant notes, an image of requested tools, and a partial listing of requested supplies;

FIG. 17B is a screenshot of a portion of an example user interface for presenting a virtual file output for an aggregated data form, wherein the portion illustrates an image of requested tools, instructions for positioning, instructions for dressing, and a partial listing of requested supplies;

FIG. 17C is a screenshot of a portion of an example user interface for presenting a virtual file output for an aggregated data form, wherein the portion illustrates an image of a requested room setup, an indication of requested music, and a partial listing of requested supplies;

FIG. 17D is a screenshot of a portion of an example user interface for presenting a virtual file output for an aggregated data form, wherein the portion illustrates a partial listing of requested supplies;

FIG. 17E is a screenshot of a portion of an example user interface for presenting a virtual file output for an aggregated data form, wherein the portion illustrates a partial listing of requested supplies;

FIG. 18A is a screenshot of a virtual file output for presenting an aggregated data form;

FIG. 18B is a screenshot of a virtual file output for presenting an aggregated data form;

FIG. 18C is a screenshot of a virtual file output for presenting an aggregated data form;

FIG. 18D is a screenshot of a virtual file output for presenting an aggregated data form;

FIG. 19 is a schematic flowchart diagram of a method for matching barcodes to medical items included on an aggregated data form;

FIG. 20 is a schematic flowchart diagram of a method for generating an aggregated data form;

FIG. 21 is a schematic flowchart diagram of a method for generating an aggregated data form;

FIG. 22 is a schematic flowchart diagram of a method for generating medical item recommendations for an aggregated data form by leveraging an artificial intelligence and/or machine learning algorithm; and

FIG. 23 is a schematic diagram illustrating components of an example computing device.

### DETAILED DESCRIPTION

Disclosed herein are systems, methods, and devices for data ingestion, unstructured file analysis, and generating aggregated data form files that can easily be updated, synced, and propagated to other systems. Additionally, disclosed herein are systems, methods, and devices for predictive modeling and suggesting items to be included in aggregated data form files based on existing inventory, existing products available in the market, historical preferences, historical inventory data, and global information regarding aggregated data forms for medical procedures.

The healthcare industry relies on preference cards when preparing for a procedure. Specifically, hospitals, clinics, and surgical centers rely on practitioner preference cards when selecting items to be present in an operating room for a procedure and when preparing a patient for the procedure. The preference card provides a listing of items that should be ready for use in the operating room before the procedure begins. The preference card may list specific pharmaceuticals, medical devices, instruments, imaging devices, personal protective equipment, and other items that must be present in the operating room for a successful procedure. The preference card may additionally include notes about how the patient should be prepared for the procedure, including how the patient should be positioned and what portions of the patient's body should be exposed for the procedure. The preference card may include notes from the practitioner about how the procedure will be performed, how many support staff should be present for the procedure, and so forth. Preference cards can be critical to good patient care and successful, safe procedures. Additionally, preference cards can be referenced when generating invoices for the procedure to ensure that all items used are accounted for.

Traditionally, preference cards include handwritten notes submitted by practitioners and stored in hard copy at each facility where the practitioner performs procedures. The preference card may be uploaded to a computer system, and still there is no efficient means for the practitioner to alter one preference card and propagate those changes to other facilities where the practitioner performs the same procedure. Additionally, there is no means for practitioners to change one preference card and then propagate that change to other preference cards for different procedures. Additionally, practitioners cannot select items to be included on a preference card based on the available inventory at a certain facility. A practitioner may unexpectedly be provided with different name brands of items, or different products, because the facility does not have the items listed on the practitioner's preference cards. Further, there is no efficient means for practitioners to share preference cards and/or provide guidance or suggestions for updating preference cards based on new products or different means of performing the procedure.

Considering the foregoing, disclosed herein are systems, methods, and devices for efficiently managing aggregated data forms across multiple independent systems. The aggregated data forms described herein may specifically be implemented in a healthcare system for storing preference card data for certain procedures. Further, disclosed herein are systems, methods, and devices for predictive modeling of available item inventory, suggesting alternative items based on historical preference data and outside data sources, and generating accurate billing and invoicing based on up-to-date information.

Before the structures, systems, and methods for generating and maintaining aggregated data forms are disclosed and described, it is to be understood that this disclosure is not limited to the structures, configurations, process steps, and materials disclosed herein as such structures, configurations, process steps, and materials may vary somewhat. It is also to be understood that the terminology employed herein is used for the purpose of describing embodiments only and is not intended to be limiting since the scope of the disclosure will be limited only by the appended claims and equivalents thereof.

In describing and claiming the subject matter of the disclosure, the following terminology will be used in accordance with the definitions set out below.

It must be noted that, as used in this specification and the appended claims, the singular forms “a,” “an,” and “the” include plural referents unless the context clearly dictates otherwise.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element or step not specified in the claim.

As used herein, the phrase “consisting essentially of” and grammatical equivalents thereof limit the scope of a claim to the specified materials or steps and those that do not materially affect the basic and novel characteristic or characteristics of the claimed disclosure.

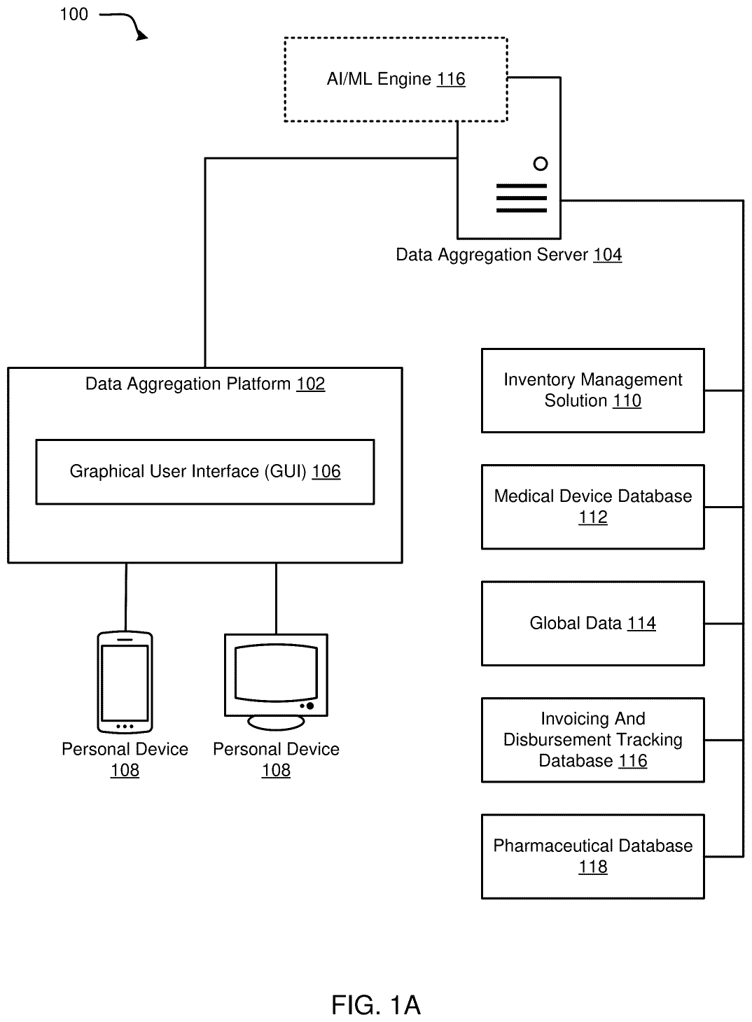

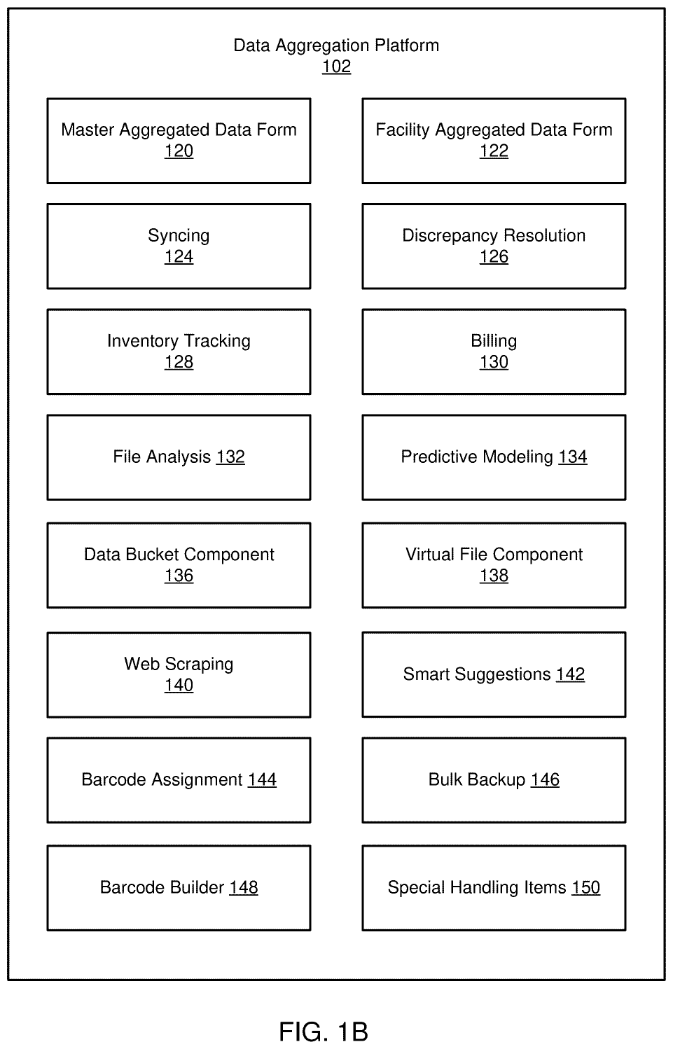

Referring now to the figures, FIG. 1A is a schematic diagram of a system for predictive modeling, file analysis, and data management. The system 100 includes a data aggregation server 104 that supports a data aggregation platform 102. The data aggregation platform 102 may be accessed by a graphical user interface (“GUI”) 106 displayed on a personal device 108. The GUI 106 may be rendered on one or more of a web browser or an application, such as an application running on a computer or mobile device. The data aggregation platform 102 is accessible by way of a personal device 108, which may include any suitable computing device such as a desktop computer, laptop computer, mobile phone, tablet, and so forth. The personal device 108 may run on an application for accessing the GUI 106 of the data aggregation platform 102.

The data aggregation server 104 ingests data from a plurality of sources, including an inventory management solution 110, medical device database 112, global data 114, invoicing and disbursement tracking database 116, and pharmaceutical database 118. The data aggregation server 104 stores, manages, and updates aggregated data forms. The aggregated data forms discussed herein may specifically be implemented to store data associated with a preference card, such as a surgical procedure preference card maintained by a healthcare facility. It should be appreciated that the aggregated data forms described herein may be implemented in other industries and implementations, and do not necessarily only include surgical preference card data.

An aggregated data form is a specialized data format comprising a plurality of data buckets. Each data bucket within the aggregated data form is associated with a certain datatype or data content. The aggregated data form stores structured information and unstructured information. In an example implementation, an aggregated data form is a manipulatable data form comprising preference card data. In this implementation, the aggregated data form may comprise text-based data buckets for “provider name,” “facility name,” “type of procedure,” “procedure name,” “items to be included at start of procedure,” and so forth. Additionally, in this implementation, the aggregated data form includes additional data buckets for receiving unstructured files, such as images, videos, audio files, emails, chat communications, and so forth. These unstructured files are stored with a specific metadata tag in associated with the aggregated data form.

The data aggregation server 104 communicates with one or more facilities (e.g., hospitals, clinics, surgical centers, and so forth), individual users (e.g., medical personnel, surgeons, hospital administrators, and so forth), and administrators by way of the data aggregation platform 102. The data aggregation server 104 generates independent accounts on the data aggregation platform 102. Each of the independent accounts is assigned to a certain user or entity and includes login information for securely accessing the data aggregation platform 102. In some implementations, one facility might have a global account on the data aggregation platform 102 that may be accessed by certain individuals, and the facility may further have individual accounts that are assigned to certain users, e.g., surgeons, healthcare personnel, administrators, surgical directors, support staff, and so forth. The data aggregation platform 102 can assign permissions for certain accounts to have read and/or write access to certain data. The data aggregation platform 102 also provides accounts to users that are not associated with a facility or other entity, and these accounts may be linked to or synced with other entities. For example, the data aggregation platform 102 enables medical personnel to create an account, enter aggregated data form preferences, and associate those aggregated data forms with certain entities, e.g., certain hospitals or surgical centers that will use the aggregated data forms.

The data aggregation server 104 includes or communicates with an artificial intelligence and/or machine learning (AI/ML) engine 116. The AI/ML engine 116 may include multiple separate AI/ML engines 116 trained to perform different tasks. The data aggregation server 104 may specifically include or communicate with an optical character recognition neural network, predictive modeling neural network, large language model, and large data model.

The AI/ML engine 116 performs optical character recognition processing to identify one or more words or text characters in an unstructured file. The optical character recognition processing techniques include text analysis and text mining. The optical character recognition process analyzes patterns of light and dark portions of an unstructured file to identify letters, numbers, and other characters. The AI/ML engine 116 recognizes characters in various fonts, so the AI/ML engine 116 is trained to correctly classify what it sees in the unstructured file. The AI/ML engine 116 identifies and classifies words and textual characters in computer-generated files (including files comprising various computer-implemented fonts) and human-generated files, including those that are handwritten. The AI/ML engine 116 identifies one or more textual characters within an unstructured file and outputs an identification and classification of those textual characters.

The AI/ML engine 116 performs predictive modeling on input data to predict future inventory of items that may be included on aggregated data forms. The AI/ML engine 116 receives historical item inventory data, global inventory data, and/or global manufacturing data to determine whether certain items might be available at a certain facility. The data aggregation server 104 additionally suggests alternative items in response to determining that a certain item is likely to be unavailable.

The data aggregation server 104 ingests data from an inventory management solution 110 that may be associated with a certain healthcare system, healthcare network, healthcare facility, wholesale distributor, manufacturer, and so forth. The inventory management solution 110 may specifically be associated with a hospital, clinic, or surgical center that provides items for use during a procedure based on information provided by the data aggregation platform 102. The inventory management solution 110 tracks the whereabouts and status of various pharmaceuticals, medical devices, and other items within a system.

The inventory management solution 110 may include sensors, storage facilities, and databases for tracking the current and future locations and status of certain items. The inventory management solution 110 may be associated with a hospital and configured to track the available quantity and the predicted future quantity of certain pharmaceuticals, medical devices, and other items used by the hospital. The inventory management solution 110 includes one or more sensors that are alerted when an item is removed from storage. The inventory management solution 110 includes a database for tracking the location and usage of certain items. For example, when an item is retrieved from storage and delivered to an operating room, the inventory management solution 110 may be notified that the item is currently within the operating room. The inventory management solution 110 stores information regarding whether certain items are immediately available or need to be sterilized, refurbished, or otherwise modified before further use. The inventory management solution 110 tracks the immediate and predicted future availability of disposable or single-use items.

The inventory management solution 110 can be an outside entity or system for inventory management. In an embodiment, the data aggregation server 104 integrates with the inventory management solution 110 by way of SFTP and the inventory management solution 110 delivers flat files at a predetermined cadence. This type of integration has the inventory management solution 110 delivering a flat file with one or more of the following details for inventory visibility, including: device identification, device type, facility name, facility identification, item identification, current inventory, minimum and maximum inventory parameters, expiration data, inventory adjustment types and details, and so forth. The data aggregation server 104 may use the inventory feed to allow users or machine learning algorithms to generate an order to replenish inventory. The data aggregation server 104 may send information to the inventory management solution 110 by way of the SFTP to facilitate restocking at an inventory device. Inventory information is at the point of purchase, as well as in other areas of the data aggregation platform 102.

The inventory management solution 110 may manage multiple locations and stock areas within an ordering location or health system. Some inventory locations utilize hardware and software to support the movement and storage of products. This creates perpetual inventory locations. The data aggregation server 104 interfaces with the hardware and/or software vendors via EDI, API, event monitors, and other means to access key information from the perpetual inventory locations. Information received may include location identifiers, drug product identifiers, current inventory quantity, maximum inventory quantity, minimum inventory quantity, average usage, stock out event, lot, and expiration date. Additionally, the data aggregation server 104 can send information to the hardware and/or software vendors to facilitate restocking inventory at the perpetual inventory device. Using this information, the data aggregation server 104 can generate recommended orders, initial recommended orders, purchase orders, and show system-wide inventory availability and usage. In an embodiment, the data aggregation server 104 has its own electronic perpetual inventory solution that can work with or independently of third-party perpetual inventory solutions and can interface with hardware vendors.

The inventory management solution 110 may include one or more robotic or automated components for counting, tracking, storing, and dispensing products. Such an inventory management solution 110 may be located at a healthcare facility such as a hospital, pharmacy, clinic, and so forth. The robotic or automated components may include a drug cabinet, drug carousel, drug refrigerator, and so forth. The robotic or automated components may include one or more scales for weighing products, scanners, image sensors, processors, and robotic arms for selecting and distributing products. The inventory management solution 110 automatically tracks the inventory that is currently available at the facility and further tracks how much of the inventory is close to expiration. The data aggregation platform 102 may be configured to automatically receive and/or retrieve real-time data from the inventory management solution 110.

In an embodiment, the data aggregation server 104 integrates with the inventory management solution 110 via SFTP and via electronic data interchange (EDI) files strictly for device replenishment. In such an embodiment, the inventory management solution 110 sends an EDI file with one or more of the following details, including: purchase order number, facility or location identifier, device identifier, item identifier, and quantity. The data aggregation server 104 may ingest the EDI file and generate a shopping cart within the data aggregation platform 102 with the necessary items. The data aggregation server 104 sends an EDI file to the inventory management solution 110 indicating any changes to the original EDI file to prepare the device for replenishment.

The data aggregation server 104 communicates with the inventory management solution 110s across a plurality of facilities and institutions. In an example implementation, the data aggregation server 104 communicates with the inventory management solution 110s for various hospitals, surgical centers, item wholesalers, and healthcare systems. The data aggregation server 104 analyzes the data received from the inventory management solution 110 to determine whether a certain item is currently in-stock and available at a certain facility, whether a certain item could be delivered to that facility from a different facility, and/or whether a certain item is likely to be in-stock and ready-for-use at a certain facility in the future. The data aggregation server 104 provides this analysis to certain accounts depending on the needs of those accounts.

The data aggregation server 104 ingests data from a medical device database 112, which may specifically include the primary medical device database managed by the Food and Drug Administration in the United States. The medical device database 112 includes information on medical device establishments, medical devices, and medical device adverse event reports. The medical device database 112 may provide comprehensive information about medical devices, including manufacturing data and barcodes for various medical devices. The data aggregation server 104 ingests information about a plurality of medical devices from the medical device database 112 by way of an API, bulk download, or web scraping.

The data aggregation server 104 may clean the raw medical device data ingested from the medical device database 112 to reduce the total memory and processing required for assessing the medical device data. The cleaning process may include removing all data columns comprising information that is not required for performing the operations of the data aggregation platform 102. The data cleaning and file size reduction may be important to ensure the data aggregation server 104 can assess the medical device data with efficient storage usage and optimal processing performance. The raw data from the medical device database 112 typically includes significant redundancy, inconsistent formatting, and unnecessary fields that can dramatically inflate file size without adding analytical value.

The cleaning process may include identifying and removing duplicate records, which is common in the medical device database 112 due to reporting inconsistencies and data submission overlaps. Duplicate detection focuses on identifying fields like MDR report numbers for adverse events or numbers for premarket notifications, while accounting for minor variations in text formatting that might mask true duplicates. Additionally, many records include empty or null fields that serve no analytical purpose, and removal of these sparse columns may significantly reduce file size. The cleaning process may further include text field optimization. The medical device database 112 typically includes verbose free-text descriptions, manufacturer narratives, and regulatory language that includes repetitive phrases and standardized formatting. Text compression techniques may be implemented to remove excessive whitespace, standardize common abbreviations, eliminate boilerplate language, and reduce storage requirements. The cleaning process includes field selection and schema optimization, which may include removing extensive regulatory metadata, submission tracking information, and administrative fields that are irrelevant to the analytical uses of the data aggregation platform 102. The cleaning process may specifically include maintaining only columns comprising information relating to device identification, dates, patient demographics, adverse event descriptions, barcode numbers, and regulatory classifications. The cleaning process may further include generating multiple separate tables for storing different aspects of the medical device data. The multiple tables may be utilized to further minimize the file size and improve the processing speeds of the data aggregation server 104 when performing typical analysis for the data aggregation platform 102.

The data aggregation server 104 ingests global data 114. The global data 114 includes information and/or analysis regarding item preferences, items associated with certain procedures, preferences of other healthcare providers, and so forth. In an implementation, the global data 114 includes information about potential shortages of certain pharmaceuticals, medical devices, and other items. The data aggregation server 104 generates item suggestions for accounts associated with the data aggregation platform 102 based on the global data 114.

The global data 114 may include information about certain medical procedures and the items associated with those medical procedures. For example, the data aggregation server 104 receives data indicating the items that are commonly used when performing a knee replacement procedure (or any other procedure). The data aggregation server 104 analyzes the data and provides suggestions to accounts on the data aggregation platform 102 that are known to perform knee replacement surgical procedures. The data aggregation server 104 provides information regarding the commonly used items, including exact commercially available versions of those items, the pricing and profit margins for those items, and the predicted availability of those items.

The data aggregation server 104 ingests data from an invoicing and disbursement tracking database 116 that includes data and pricing model for items and procedures that may be included on aggregated data forms. The invoicing and disbursement tracking database 116 may include an independent server and/or database, or the invoicing and disbursement tracking database 116 may be incorporated within the data aggregation server 104. The data aggregation server 104 may communicate with the invoicing and disbursement tracking database 116 by way of an Application Program Interface (API) or other secure means of communication. The invoicing and disbursement tracking database 116 provides up-to-date information on pricing for certain items and services. The invoicing and disbursement tracking database 116 may be facility-specific or may include industry-wide pricing.

The invoicing and disbursement tracking database 116 may include an internal pricing model associated with a facility such as a healthcare system, a healthcare network, a hospital, a surgical center, a clinic, and so forth. The invoicing and disbursement tracking database 116 includes real-time pricing information about items that may be included on aggregated data forms and may further include pricing information for generic or alternative items. The invoicing and disbursement tracking 116 may indicate the facility's cost for certain items and may further indicate the facility's pricing for billing those items to patients. The invoicing and disbursement tracking database 116 may indicate the profit margins for various items. The data aggregation server 104 assesses the pricing and billing information and may provide suggestions to providers regarding the most cost-effective items, the items with the greatest profit margin, and so forth.

The data aggregation server 104 ingests data from a pharmaceutical database 118, which may specifically include the approved drug products database managed by the Food and Drug Administration in the United States. The pharmaceutical database 118 includes information about prescription and over-the-counter drugs, with therapeutic equivalence rations. The data aggregation server 104 may ingest data from the pharmaceutical database 116 by way of an API integration, bulk download, direct download, or FAERS data integration.

The data aggregation server 104 may clean the raw pharmaceutical data ingested from the pharmaceutical database 118 to reduce the total memory and processing required for assessing the medical device data. The cleaning process may include removing all data columns comprising information that is not required for performing the operations of the data aggregation platform 102. The data cleaning and file size reduction may be important to ensure the data aggregation server 104 can assess the medical device data with efficient storage usage and optimal processing performance. The raw data from the pharmaceutical database 118 typically includes significant redundancy, inconsistent formatting, and unnecessary fields that can dramatically inflate file size without adding analytical value. The cleaning process for the raw pharmaceutical data may be similar to the cleaning process described for the raw medical device data, as described above.

FIG. 1B is a schematic block diagram illustrating potential components and functionalities of the data aggregation platform 102. The data aggregation platform 102 may specifically be utilized to generate aggregated data forms for storing surgical preference card information, including a listing of medical items to be included in an operating room, a description of how those medical items should be arranged, a description of how the room should be prepared for the surgeon, a description of which personnel should be present for the surgical procedure, and so forth.

As described herein, an aggregated data form for a surgical preference card may include a plurality of medical items, and each medical item describes a type of medical device or pharmaceutical, to be present for a surgical procedure. The medical item may be a “generic” term that refers to a type of medical device, and the medical item may be satisfied by a plurality of various medical device products offered by various manufacturers with different packaging and so forth. The medical item may similarly be a “generic” term that refers to a type of pharmaceutical, and the medical item may be satisfied by a plurality of various name-brand and generic drug products offered by various manufacturers with different packaging and so forth. The data aggregation server 104 manages the listings of medical items within each aggregated data form and automatically identifies actual medical device products and drug products to satisfy the requested medical items.

The data aggregation platform 102 manages the generation and storage of master aggregated data forms 122 and facility aggregated data forms 124. In some implementations, a master aggregated data form is a master preference card associated with a plurality of healthcare facilities, and a facility aggregated data form is a facility preference card associated with a specific facility. It should be appreciated that the aggregated data forms may apply in other industries and circumstances, and do not necessarily include preference card data.

The master aggregated data form 120 component generates and manages aggregated data forms that are not specifically associated with a healthcare facility. The facility aggregated data form 122 components generates and manages aggregated data forms that are associated with a certain facility. The master aggregated data form may be associated with a certain healthcare practitioner or group of healthcare practitioners. A master aggregated data form is assumed to reflect the healthcare practitioner's general preferences for the referenced procedure. When the aggregated data form is associated with a facility, it is then referred to as a facility aggregated data form and represents the healthcare practitioner's preferences when performing the specified procedure at that facility.

The data aggregation platform 102 provides an account associated with a healthcare practitioner. The healthcare practitioner inputs information into the data aggregation platform 102 including, for example, the user's name, healthcare specialty, healthcare network associations, facility associations, healthcare insurance associations, and so forth. The user further indicates the names and/or billing codes for certain procedures performed by the healthcare practitioner, and where the healthcare practitioner performs those procedures. The user may generate an independent aggregated data form for each procedure (these are referred to as master aggregated data forms). The user may further generate in independent aggregated data form for each procedure at certain facilities (these are referred to as facility aggregated data forms). In an example implementation, the user is an orthopedic surgeon, and the user generates an independent master aggregated data form for each procedure the user performs. The user may further generate facility aggregated data forms that indicate different protocols or items the user will use when performing the procedure at a certain facility.

In some implementations, the responsibility to update a facility's aggregated data form falls on the users who relate to that facility, whereas the responsibility to update master aggregated data forms falls on the healthcare practitioner associated with those master aggregated data forms. The facility and/or healthcare practitioner may provide access to aggregated data forms for other persons/accounts to access and update the aggregated data forms on their behalf. Changes will be made to master aggregated data forms and to facility aggregated data forms over time, and these changes are propagated throughout the data aggregation platform 102. The master aggregated data forms 122 are linked to the facility aggregated data forms 124, and vice versa. If the healthcare practitioner implements a change to a master aggregated data form, the healthcare practitioner may choose to push that change out to one or more facilities associated with the master aggregated data form.

When a facility aggregated data form is linked to a master aggregated data form, changes made against the one are propagated as suggested changes to the other. Facility aggregated data forms may be linked to one master aggregated data form, but master aggregated data forms may be linked to multiple facility aggregated data forms, even within the same facility. This allows for changes made by a facility to be floated up to the practitioner's master aggregated data form (and approved by the practitioner), and then proposed as changes to other facility aggregated data forms associated with the same master aggregated data form.

The master aggregated data form 120 and facility aggregated data form 122 components are initiated when a user changes or adds contents to an aggregated data form. This can include generating a new aggregated data form, adding an item, removing an item, changing a quantity of an item, adding, editing, or removing a note, and so forth. A healthcare practitioner may generate a new master aggregated data form at any time. The master aggregated data form has access to “public” inventory, including a listing of items that are maintained and scrubbed regularly by the data aggregation platform 102. This includes, for example, pharmaceuticals, gloves, equipment, imaging devices, robotic devices, computing systems, and so forth. When a new master aggregated data form is created, each facility associated with the healthcare practitioner is notified and given the opportunity to create a linked facility aggregated data form that captures the changes made to the master aggregated data form to stay up to date with the practitioner's preferences.

The data aggregation platform 102 supports one or more user accounts associated with a facility, which may be referred to herein as facility users. A facility user may log on to the data aggregation platform 102 and view a list of practitioners that are associated with the facility. The facility user can see a list of the master aggregated data forms and/or facility aggregated data forms that the facility has for each practitioner. Each facility aggregated data form may be linked to a single master aggregated data form, but more than one facility aggregated data form may be linked to the same master aggregated data form. When the facility creates a facility aggregated data form without a master aggregated data form, the practitioner is offered the opportunity to create a master aggregated data form and link the master aggregated data form to the facility aggregated data form. Otherwise, the practitioner may link the new facility aggregated data form to an existing master aggregated data form. The facility user may also have permission to create a new facility aggregated data form and preemptively link that card to a master aggregated data form.

The data aggregation platform 102 manages the deletion of master/facility aggregated data forms. When a master aggregated data form is deleted, the data aggregation server 104 checks for linked facility aggregated data forms. If a facility aggregated data form is synced to the master aggregated data form that is pending deletion, then practitioner may select a new master aggregated data form to link to the facility aggregated data forms and/or unlink the facility aggregated data forms to the master aggregated data form. The data aggregation platform 102 updates the links on the facility aggregated data forms to the specified master aggregated data form and begins the discrepancy resolution 126 process. In the case of selecting to unlink the facility aggregated data forms, the data aggregation platform removes the links that exist to the facility aggregated data forms for that practitioner, notifies the facilities that have been removed, and provides the facilities the opportunity to correct the issue by linking a facility aggregated data form to the new master aggregated data form.

When a facility aggregated data form is deleted, the data aggregation platform 102 notifies the practitioner that the action has occurred. The data aggregation platform 102 gives the practitioner the opportunity to delete the associated master aggregated data form. This is potentially gated on whether there are other facility aggregated data forms linked to the master aggregated data form at the time.

The syncing 124 component manages push notifications and data syncing across different systems, platforms, facilities, and user accounts. The syncing 124 component may receive an indication that a user modified a master aggregated data form. The syncing 124 component identifies all facility aggregated data forms associated with the master aggregated data form. The syncing 124 component pushes the changes to each of the facility aggregated data forms and may further generate a notification for each of the associated facilities to notify the facilities of the change.

The syncing 124 component manages links between facility aggregated data forms and master aggregated data forms. At times, the data aggregation platform 102 needs to link an existing facility aggregated data form to a master aggregated data form. This may specifically happen when a new system or new facility-user system is set up. This may also occur when the master aggregated data form is modified. When a link is created, the syncing 124 components looks at the differences between the master aggregated data form and the facility aggregated data form. This includes mapping items between inventories, correcting discrepancies in quantities, updating notes, and other conflict resolution processes that occur when comparing the two aggregated data forms. When an item in an aggregated data form is added, changed, or removed, the card's Last Updated Timestamp is updated. This will trigger out-of-sync notifications to linked aggregated data forms.

The syncing 124 component notifies a facility user when there are changes that have been made on an aggregated data form linked to the facility. Theo facility user may click in to review the changes, and the user interface will populate a list of selected changes to be made to the card. The facility user can review the proposed changes, remove changes, modify changes, and add additional changes. When the facility user is satisfied, the facility user may submit the changes to the target aggregated data form and the relationship will be marked as synced as of the time the batch of changes was submitted. The application or rejection of proposed changes is referred to as “syncing the cards” The aggregated data form includes a “Last Updated” field that indicates a timestamp of the most recent sync cycle.

The syncing 124 component includes a change suggestion engine. The change suggestion engine receives a proposed change and a target card associated with the proposed change. The change suggestion engine suggests what may be done to apply a comparable change to the target card. The change suggestion engine suggests take numerous factors into account when making suggestions, including item names, quantities, types, previously accepted suggestions, rejected suggestions, relationship weights, and so forth. The change suggestion engine proposes a specific action, such as adding a newly created item, editing an existing aggregated data form, editing an existing line-item on an aggregated data form, and so forth. The user may provide feedback, and this feedback is consumed as training data for training the change suggestion engine to make better suggestions. The change suggestion engine may incorporate an AI/ML engine.

The discrepancy resolution 126 component identifies and resolves differences between master aggregated data forms and linked facility aggregated data forms. The discrepancy resolution 126 component resolves differences between linked aggregated data forms. The discrepancy resolution 126 copies from changes from one aggregated data form to another aggregated data form, including items mapped between the cards and quantities of items. The discrepancy resolution 126 component additionally creates a set of changes that would be necessary to bring the aggregated data forms into alignment.

In evaluating the changes, the discrepancy resolution 126 component assesses item mappings that already exist for the facility. If a user has already linked a public item to a facility item, the discrepancy resolution 126 maintains that mapping when comparing the master aggregated data form to the facility aggregated data form. This item mapping allows the discrepancy resolution 126 to recognize matches between items that may not be obvious based on the item names. The data aggregation platform 102 displays a list of changes that have not yet been resolved and allows those changes to be propagated or removed.

For each source action, the discrepancy resolution 126 component defines default behaviors for the target action. Unless otherwise specified, the default action is No Action. When a note is added to an aggregated data form, the discrepancy resolution 126 component checks whether the note's title already exists. If so, the discrepancy resolution 126 component suggests appending the text of the new note. If there are no notes with the same title, the discrepancy resolution 126 component suggests creating a new note with the text from the linked card. When a note is removed from an aggregated data form, the discrepancy resolution 126 component checks for a note that is either mapped to that note or has the same title. If a duplicate note exists, the discrepancy resolution 126 component suggests deleting the duplicate note.

When a note is updated, the discrepancy resolution 126 component checks for a mapped note or a note with the same title. If such a note exists, the discrepancy resolution 126 component applies the delta to the note's text. If that is successful, the discrepancy resolution 126 component suggests automatically updated the note with the updated text. If it is unsuccessful, the discrepancy resolution 126 requests manual intervention for a user to update the note and presents the original text and permits the user to manually update the text of the note. If there is no mapped note, the discrepancy resolution 126 component suggests that the note be added and provides a user the option to edit the note title or note text.

When an item is added to a master aggregated data form and/or facility aggregated data form, the discrepancy resolution 126 component determines whether there is an item mapping to the target context (i.e., public context or facility context).

The inventory tracking 128 component communicates with one or more inventory management solution 110s and may additionally communicate with one or more of invoicing and disbursement tracking 116, global data 114, or item medical device database 112 systems. The inventory tracking 128 component provides suggestions to users based on historical, current, or predicted future inventory at a facility. In an example implementation, the inventory tracking 128 component populates a master aggregated data form and/or facility aggregated data form with items that are known to be available at a certain facility and may additionally provide suggestions for alternate items.

The inventory tracking 128 component additionally communicates with the item medical device database 112 to identify specific items that may be included on an aggregated data form. The items may include pertinent information such as manufacturer, cost, supplier, and so forth.

The data aggregation platform 102 exists in at least two contexts, including the public context and the facility context. Items exist within their context (i.e., the public context and/or the facility context) and have data that is specific to that context. The data can be mapped to an item in another context. Any number of public items may be mapped to any number of facility items. Procedure names and procedure billing codes are also mappable items. Item mapping includes a scope. The data aggregation platform 102 maps an item from the public context to the facility context when dealing with a change to an aggregated data form. A user can elect to have the mapping apply only to a certain aggregated data form or to create a more general, facility-level mapping. The data aggregation platform 102 floats card-level mapped items to the top of a drop-down list, followed by facility-level mapped items, followed by non-mapped items from the target context.

The billing 130 component calculates and/or suggests the materials cost for a procedure based on items included on an aggregated data form. The billing 130 component may communicate with outside systems and databases such as the invoicing and disbursement tracking database 116. The billing 130 component may suggest items based on the wholesale cost and/or the profit margin for billing the use of that item during a procedure. The billing 130 component may provide suggestions to a practitioner user and/or facility user regarding similar items that might reduce cost while providing the same functionality.

The pricing information consumed by the billing 130 component may be retrieved from a public catalog compiled from multiple sources. Each facility in communication with the data aggregation server 104 may additionally provide more accurate pricing for individual items. In some cases, billing is done per-procedure rather than in an itemized fashion for each item used during the procedure. When this is the case, every item wasted comes directly from the bottom line for the procedure. The billing 130 component communicates with the inventory management solution 110 and invoicing and disbursement tracking database 116s at the facility to retrieve this information and itemize all items used during the procedure. The billing 130 component may communicate with the inventory management solution 110 and/or invoicing and disbursement tracking database 116s by way of HL7 or other communication standards.

The billing 130 component collects equivalent item designations and recommends using a different item with a lower cost when applicable. The billing 130 component tracks when items are replaced on an aggregated data form and determines whether the item was replaced with an equivalent item. The billing 130 component additional tracks variant pricing between practitioners at certain facilities and across different facilities.

The file analysis 132 is executed by the AI/ML engine 116 to identify objects of interest and textual characters in unstructured data. An unstructured file (or unstructured data) includes information that does not have a pre-defined data model or is not organized in a pre-defined manner. Unstructured files may be human generated, or machine generated. Examples of unstructured files includes, for example, audio files, video files, images, Microsoft© Word documents, Microsoft® PowerPoint®, emails, chat message logs, data from social networking sites, text messages, locations, call recordings, portable document format (PDF) files, images or scans of hardcopy documents, and so forth. The file analysis 132 may include one or more independent AI/ML engines that are each trained to perform different types of files analysis. The file analysis 132 may include an AI/ML engine trained to identify and/or classify objects of interest within image or video data. The file analysis 132 may include an AI/ML engine trained to identify and/or classify words or music recorded in audio data. The file analysis 132 may include an AI/ML engine trained to identify textual characters and words in an image, scan, video stream, or other form of unstructured data.

The file analysis 132 includes a machine learning algorithm trained to execute optical character recognition processes to identify one or more words or textual characters in an unstructured file. Textual characters include letters, numbers, punctuation characters, emojis, and other characters. The file analysis 132 is trained to “read” an unstructured file to identify textual characters and/or words within the file, and further to classify the content of the textual characters and/or words within the file.

The file analysis 132 performs optical character recognition to identify one or more textual characters and/or words within an unstructured file. In an example implementation, the unstructured file is an image or a scan of a hardcopy surgical preference card. The hardcopy surgical preference card may include handwritten characters and/or computer-printed characters. The file analysis 132 analyzes the image/scan of the hardcopy surgical preference card and identifies, for example, the name of the surgeon associated with the preference card, the name of the facility associated with the preference card, the name of the surgical procedure, the SKUs and descriptions of the items to be included in the surgical operating room, the quantities of each item to be included in the surgical operating room, the surgeon's preference for certain music to be played during the procedure, and so forth.

Optical character recognition automatically analyzes printed and/or handwritten textual characters and translates those characters into a form that a computer can process and understand. Optical character recognition includes the process of turning a picture or scan of text into text itself, or in other words, translating an image (or other unstructured data file) into a text file, such as a TXT or DOC file. The file analysis 132 is trained on a plurality of vast datasets comprising different fonts, different types of handwriting, different languages, different textual characters, and so forth.

The predictive modeling 134 is performed by the AI/ML engine 116 to predict the current and future inventory of certain medical items, including pharmaceuticals and medical devices. The predictive modeling 134 component receives information from invoicing and disbursement tracking 116, global data 114, item medical device database 112, and/or inventory management solution 110 and predicts the current and future inventory of certain pharmaceuticals, medical devices, and other items. The predictive modeling 134 component may include an AI/ML engine trained to identify patterns in item availability and to predict future item availability based on those patterns.

The predictive modeling 134 component may predict whether a certain item will be in-stock and available for immediate use at a certain facility at a time in the future. This analysis is based on historical availability for that item at that facility and may further be based on one or more of the date of the procedure (including the day of week of the procedure), the time of the procedure, the department within the facility where the procedure will be performed, and so forth. The predictive modeling 134 component may predict that the item is unlikely to be available for use at the time of the scheduled procedure. If this is the case, the predictive modeling 134 component may further provide a suggested alternative item that will likely be available at the time of the schedule procedure.

The predictive modeling 134 component predicts future procedures to be performed based on past trends. These predictions consider the seasonality of procedures where significant, for example where certain procedures are more likely to be performed at certain times of the year, certain weeks of a month, certain days of the week, and so forth. The predictive modeling 134 component analyses past procedure information and item usage from those past procedures. The predictive modeling 134 components predicts how many of each item will be used during the procedure. This is a deviation from assuming that each item that was pulled and prepared for the procedure was in fact used during the procedure.

The predictive modeling 134 component may include an analysis of variance (ANOVA) statistical model. ANOVA is a collection of statistical models and their associated estimation procedures used to analyze the differences among means. ANOVA is based on the law of total variance, where the observed variance in a particular variable is partitioned into components attributable to different sources of variation.

The predictive modeling 134 component may include a long short-term memory (LSTM) artificial AI/ML engine architecture. LSTM is an artificial recurrent AI/ML engine. Unlike standard feedforward AI/ML engines, LSTM has feedback connection. The LSTM architecture can process single data points (such as images) and can further process sequences of data (such as speech or video). The LSTM architecture includes a cell, an input gate, an output gate, and a forget gate. The cell remembers values over arbitrary time intervals and the three gates regulate the flow of information into and out of the cell.

The predictive modeling 134 may include a recurrent AI/ML engine (RNN) architecture. The RNN architecture may be particularly implemented for modeling upcoming procedures and predicting future item usage based on past procedures. The RNN architecture is a class of artificial AI/ML engines where connections between nodes form a directed graph along a temporal sequence. This allows the RNN to exhibit temporal dynamic behavior. RNNs can use an internal state (memory) to process variable length sequences of inputs.

The data bucket component 136 assigns data to certain data buckets associated with the aggregated data form. The aggregated data form includes a plurality of data buckets, wherein each data bucket is intended to be associated with data of a certain type and content. In an example implementation, the aggregated data form is a surgical preference card. In this implementation, the aggregated data form may include one data bucket intending to receive one or more images of an example surgical setup and/or operating room. The aggregated data form may additionally include a plurality of data buckets intending to receive textual data regarding, for example, the name of the surgeon or facility, the type of procedure to be performed, the surgeon's preferences, the pharmaceuticals to be present in the operating room, the medical devices to be present in the operating room, and so forth. The aggregated data form may additionally include a plurality of data buckets intending to receive data regarding real-time pricing for medical devices and/or pharmaceuticals, and these data buckets may be in communication with an API or other communication protocol associated with, for example, a manufacturer, distributor, central healthcare system, internal facility inventory management solution, and so forth.

The data bucket component 136 may receive an output from the AI/ML engine 116 comprising one or more textual characters and/or words that were extracted from an unstructured file. The data bucket component 136 may then take those textual characters and/or words and assign them to individual data buckets associated with the aggregated data form. For example, if the aggregated data form is a preference card, then the data bucket component 136 may receive a plurality of textual outputs from the AI/ML engine 116 that would read from an image, scan, PDF, etc. of an existing preference card. The data bucket component 136 receives these textual outputs and classifies and/or assigns them to a data bucket within the aggregated data form.

For example, the AI/ML engine 116 may output a name, such as “Dr. Gregory Smith,” and then the data bucket component 136 will assign that textual output to the “surgeon name” data bucket. Further for example, the AI/ML engine 116 may output a phrase, such as “knee arthroscopy,” and then the data bucket component 136 will assign that textual output to the “procedure name” data bucket. Further for example, the AI/ML engine 116 may output a phrase, such as “torniquet machine,” and then the data bucket component 136 will assign that textual output to an “equipment” data bucket, which comprises a listing of equipment to be present in the operating room during the knee arthroscopy procedure performed by Dr. Gregory Smith, and so forth.

The data bucket component 136 may be automated to classify the content of certain words and phrases. In an example use-case, the data aggregation platform 102 is implemented to aggregate data and maintain data in surgical preference cards. In this example, the data bucket component 136 may consume words and phrases that are known to have significance within a medical vocabulary. For example, each procedure has a name, each medical device has a name, each pharmaceutical has a name, and so forth. When new names and phrases are created, and when new procedures are invented, the data bucket component 136 checks those new names and phrases against known words, phrases, procedure names, medical devices, pharmaceuticals, and so forth. The data bucket component 136 predictively identifies whether the new words or phrases should be classified as, for example, a surgeon, procedure, pharmaceutical, medical device, and so forth. Thus, the data bucket component 136 identifies certain words, phrases, and blocks of text as belonging to a certain data bucket based on the nature of the content, regardless of where those words, phrases, or blocks of text were located on the page of the unstructured file that was analyzed by the AI/ML engine 116.

The virtual file component 138 generates a virtual file output based on data stored in the aggregated data form. The virtual file output may include a formatted electronic document that may be rendered on a graphic user interface of an electronic device or printed in hardcopy format. The virtual file output may include a rendered user interface comprising a web-accessible and editable layout for viewing and manipulating data within the aggregated data form. The virtual file output may include interchangeable data representations, for example, data in JSON or other platform-agnostic structure of data such as binary, XML, and so forth. The virtual file output may include a web-accessible read-only version of the data stored in the aggregated data form, wherein this read-only version of the data may be accessible without authentication. The virtual file output may include a QR code (or other unique code) comprising a URL describing the location of the aforementioned read-only version of the data.

In an example implementation, the virtual file output is a collection of data stored on a server, and this collection of data is accessible on a unique web URL associated with that aggregated data form. In a further example implementation, the virtual file output is a single document, such as a Microsoft® Word document, PDF, video file, image, or another document. In a further example implementation, the virtual file output is a message, such as an email, push notification, audio message, or other communication that communicates the content stored in associated with the aggregated data form.

The web scraping 140 may be executed by an external third-party service in communication with the data aggregation server 104 and/or may be executed by the data aggregation server 104 itself. The web scraping 140 performs data extraction for certain types of data across certain web pages. The web scraping 140 may extract structured data from publicly accessible webpages, and from private webpages necessitating a secure login or connection. The web scraping 140 utilizes a strategic approach that balances data acquisition needs with respect for website terms of service and rate limiting considerations.

The web scraping 140 (may alternatively be referred to as a “data scraping” component) identifies, extracts, and saves data retrieved from webpages. The web scraping 140 additionally “reads” that data and classifies the data based on its content. In an implementation, the web scraping 140 additionally includes a discrepancy resolution 126 component to determine whether newly retrieved data should replace data that is currently stored on the data aggregation server 104. For example, the web scraping 140 may identify an updated name, SKU, or other information for a medical device that is stored in associated with the data aggregation platform 102. The discrepancy resolution 126 component then determines whether the currently stored information regarding the medical device should be replaced with the new information extracted by the web scraping 140.

The web scraping 140 may communicate directly by way of Hypertext Transfer Protocol (HTTP) and may provide a script execution environment to simulate user interaction. The web scraping 140 may utilize a web browser process to perform the HTTP communication and execute web scripts designed to render the HTML page for processing. In this case, the web scraping 140 may execute the scripts it pulls rather than merely receive those scripts as text. This may be necessary on some web pages to enable the web scraping 140 to convince those web pages that the web scraping 140 is not a bot, because the scripts are running in a web browser.

The web scraping 140 caches the content of the web pages its reads and performs additional scraping tasks against those web pages without requiring additional trips to the source web server. This reduces the network traffic and load on the server and may be necessary to obtain the information or to extract additional data.

The smart suggestions 142 is executed by the AI/ML engine 116 to provide suggested amendments to an aggregated data form. The smart suggestions 142 is trained on historical aggregated data forms for a plurality of different surgeons, surgical facilities, and surgical procedures. The smart suggestions 142 is further trained upon actual usage data for historic surgical procedures, current inventory data, and historical inventory data for medical items at various healthcare facilities. The smart suggestions 142 assesses an aggregated data form and provides suggested amendments to the aggregated data form. The suggested amendments may include, for example, amending a quantity of a certain medical item, identifying a certain medical device product and/or pharmaceutical drug product that satisfies a medical item on the aggregated data form, and/or suggesting additional medical items to be included in the aggregated data form.

The smart suggestions 142 implements the AI/ML engine 116 for surgical preference card optimization to significantly improve surgical efficiency, reduce waste, and enhance patient outcomes through data-drive recommendations. The smart suggestions 142 relies on comprehensive data integration from multiple sources, including historical usage patterns from electronic health records and/or inventory management solutions, comparative analysis across similar surgical procedures and surgeon preferences, real-time inventory levels and availability constraints, and outcome metrics that correlate supply choices with surgical success rates and complication frequencies.

The AI/ML engine 116 architecture employes multiple complementary algorithms to address different aspects of preference card optimization. Collaborative filtering techniques, similar to those used in recommendation systems, identify patterns where surgeons performing similar procedures benefit from specific medical items that a target surgeon has not yet adopted. Association rule mining algorithms discover frequently occurring medical items across a plurality of aggregated data forms associated with a certain facility and/or surgical procedures. Tiem series forecasting models predict supply needs based on seasonal patterns, surgeon scheduling, and historical usage trends. Classification algorithms categorize surgical cases to ensure recommendations align with specific procedure requirements and patient characteristics for an aggregated data form.

The smart suggestions 142 provides efficiency recommendations, including recommending an increase or reduction in the quantity of a medical item that is present within an operating room for a surgical procedure. This may be calculated based upon a determination that a portion of the medical item is typically unused when performing a certain surgery. This may be calculated based upon a determination that more of the medical item often or sometimes needs to be acquired while the surgery is ongoing. This may be calculated based upon a determination that many surgeons across different facilities use a different quantity of the medical item when performing the same procedure or a similar procedure.

The barcode assignment 144 assigns one or more possible barcodes to each medical item listed on an aggregated data form. Each aggregated data form may include a plurality of medical items, and each of the medical items refers to a type of device or drug. In many cases, a medical item may be satisfied by many different medical device products or pharmaceutical drug products that are made by different manufacturers or presented in different packaging, and so forth. Each one of these various medical device products or pharmaceutical drug products is assigned a different barcode, and thus, each medical item on the aggregated data form may be associated with multiple possible barcodes. The barcode assignment 144 assigns the one or more possible barcodes (i.e., the one or more possible medical device products or pharmaceutical drug products) associated with each medical item on the aggregated data form.

The barcode assignment 144 executes a data matching and standardization process that bridges the gap between manufacturer product identification systems, hospital inventory management, and the aggregated data forms. The barcode assignment 144 is based upon establishing data linkages between data entries ingested from the medical device database 112 and/or pharmaceutical database 118, and the medical items listed in the aggregated data forms. This may further include establishing data linkages between the data entries ingested from the medical device database 112 and/or pharmaceutical database 118, and the data entries ingested from the inventory management solution 110 for a facility. The matching algorithm accounts for variability in how medical items are described across different data sources and organization systems. The barcode assignment 144 may leverage the AI/ML engine 116 to perform fuzzy string matching, natural language processing for medical terminology, and execute similarity scoring algorithms to identify matches between preference card medical items, medical device database 112 entries, pharmaceutical database 118 entries, and inventory management solution 110 entries.

The bulk backup 146 enables a user to download a compressed or uncompressed file comprising all aggregated data forms associated with a certain facility, surgeon, or surgery type. The bulk backup 146 output file may be stored locally on a computer system located at a surgical facility. This enables practitioners to access all aggregated data form data in the event of a network outage.

The barcode builder 148 enables a user to build an aggregated data form by scanning the barcodes of various medical devices and/or pharmaceuticals. The barcode builder 148 adds each scanned medical device and/or pharmaceutical to the aggregated data form in response to a user scanning the barcode with a barcode scanner while building the aggregated data form on the data aggregation platform 102. The barcode builder 148 may further “genericize” each scanned medical device and/or pharmaceutical to refer to the corresponding medical item type that could be satisfied by various products sold by various manufacturers.

The special handling items 150 incorporates specially handled medical items into the aggregated data forms, including, for example, consigned goods, loaned items, inventory requiring detailed tracking, items provided by an in-person medical device or pharmaceutical representative, and so forth. Specially handled items may specifically include medical items that are prohibitively inexpensive to be owned within a facility's inventory, and may include items that are delivered to a surgery by a sales representative. The special handling items 150 may include providing contact information for a sales representative that must provide specially handled items for a certain surgical procedure. In an example implementation, this may include an orthopedic medical device sales representative that arrives in-person to provide various sizes of medical implants for an orthopedic surgery. The special handling items 150 may export information to a patient's health record to ensure data is maintained regarding the size and type of item that was provided to or used on the patient during the surgical procedure. The special handling items 150 attaches metadata fields to certain items within the aggregated data form to trigger conditional logic for specialty handling of those items by other teams at the facility, including, for example, accounting teams and external purchasing teams.

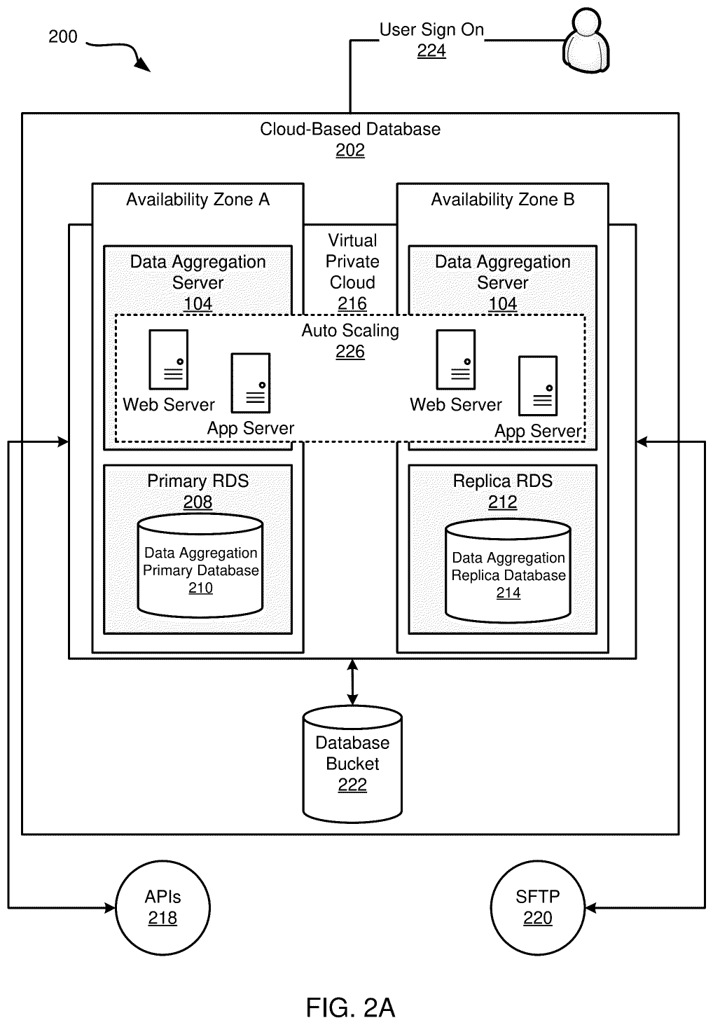

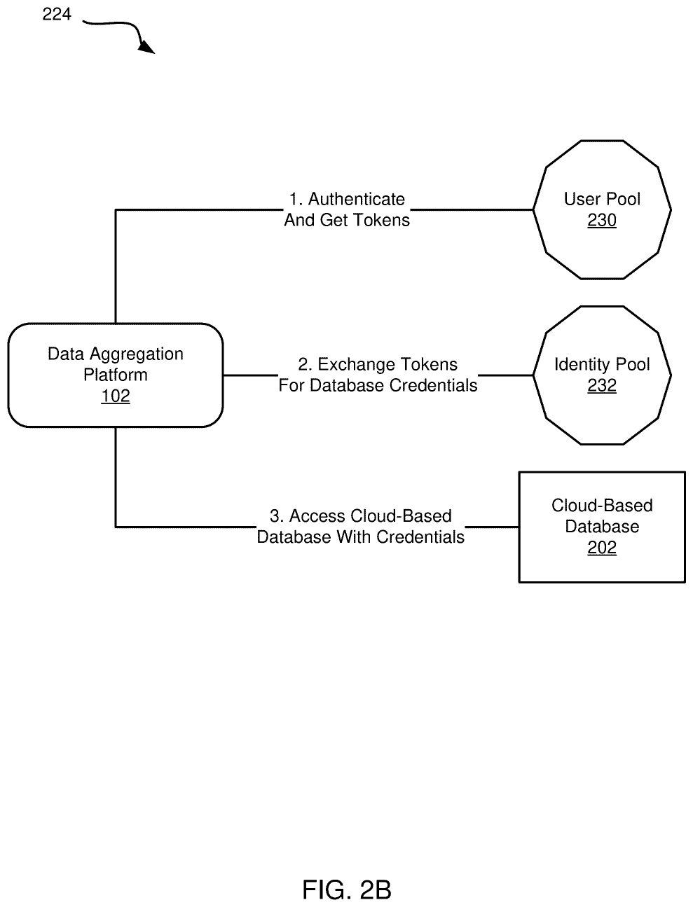

FIG. 2A is a schematic block diagram of a system 200 for storing and managing data, such as the systems associated with the data aggregation server 104. The system 200 illustrated in FIG. 2A may be implemented in conjunction with the system 100 illustrated in FIG. 1A. The system 200 includes a cloud-based database 202 supporting the data aggregation server 104. The cloud-based database 202 includes an Availability Zone A and an Availability Zone B. The Availability Zone A includes a first instance of the data aggregation server 104 and the Availability Zone B includes another instance of the data aggregation server 104. Each of the instances of the data aggregation server 104 includes a web server and an app server, and the cloud-based database 202 auto-scales the processing and storage resources between the web servers and app servers for the Availability Zone A and the Availability Zone B. The Availability Zone A includes a primary relational database service (RDS) 208 and the Availability Zone B includes a replica relational database service 212. The data aggregation primary database 210 is stored on the primary relational database service 208 and the data aggregation replica database 214 is stored on the replica relational database service 212. The virtual private cloud 216 of the cloud-based database 202 communicates with outside parties by way of Application Program Interfaces 218 and Secure File Transfer Protocol (SFTP) 220 messaging. The cloud-based database 202 includes a database bucket 222 for storing information associated with the data aggregation platform 102. Users interacting the data aggregation platform 102 can sign on 224 to the service by communicating with the cloud-based database 202.

The cloud-based database 202 includes processing and storage resources in communication with the network 120. The cloud-based database 202 includes a resource manager for managing the usage of processing and storage resources. The resource manager of the cloud-based database 202 performs auto scaling 226 load balancing to ensure adequate processing and storage resources are available on demand based on real-time usage.

The availability zones represent discrete datacenters with redundant power, networking, and connectivity for supporting the data aggregation server 104. The availability zones enable the ability to operate production applications and databases in a more highly available, fault tolerant, and scalable way than would be possible with a single datacenter. The Availability Zone A and Availability Zone B are interconnected with high-bandwidth, low-latency networking, over fully redundant, dedicated metro fiber providing high-throughput, low-latency networking between the availability zones. All traffic between the availability zones is encrypted. The network performance of the availability zones is sufficient to accomplish synchronous replication between the availability zones. Applications, modules, components, and processing methods can be partitioned between the availability zones of the cloud-based database 202. When applications are partitioned across the availability zones, the data aggregation server 104 operates with increased protection and isolation from outages that may be caused by a low in power, hardware issues, software issues, and so forth. The availability zones are physically separated by a meaningful geographic distance to ensure the hardware supporting the availability zones will not be impacted by the same outside forces, such as power outages, natural disasters, and so forth.

The virtual private cloud 216 is an on-demand configurable pool of shared resources allocated within the cloud-based database 202. The virtual private cloud 216 provides isolation between different users communicating with the cloud-based database 202, e.g., different facilities, user accounts, and clients in communication with the data aggregation platform 102. The isolation between one virtual private cloud 216 user and all other users of the same cloud is achieved through allocation of a private IP subnet and a virtual communication construction such as a VLAN or a set of encrypted communication channels per user. The virtual private cloud 216 provides isolation between users within the cloud-based database 202 and is accompanied with a VPN function allocated per-user within the virtual private cloud 216. This secures the remote access to the data aggregation platform 102 by way of authentication and encryption. The data aggregation platform 102 is then essential run on a “virtually private” cloud, even if the processing and storage resources are provided by a third-party cloud-based database service, such as Amazon Web Services®.

The auto-scaling 226 is performed by a resource manager of the cloud-based database 202. The resource manager distributes workload between the web servers and the app servers of the various availability zones of the cloud-based database 202. In some cases, one client of the data aggregation platform 102 may consume a large quantity of storage resources and processing resources at a certain time, and the resource manager will allocate different web servers and app servers across the availability zones to ensure the client receives an adequate quantity of storage and processing resources. The auto-scaling 226 is performed in real-time to meet the needs of the data aggregation platform 102.

The primary and secondary relational database services 208, 212 provide a means to access, replicate, query, and write to the data aggregation database instances 210, 214. The data aggregation primary database 210 may include a copy of data associated with the data aggregation platform 102. The data aggregation replica database 214 may include a replica copy of all or some of the data stored on the data aggregation primary database 210. The replicated databases provide fault-tolerance and protect the data aggregation platform 102 from becoming inoperative during a power outage, hardware outage, or natural disaster.

The database bucket 222 provides object storage through a web service interface. The database bucket 222 uses scalable storage infrastructure that can be employed to store any type of object. The database bucket 222 may store applications, software code, backup and recovery, disaster recovery, data archives, data lakes for analytics, and hybrid cloud storage to support the data aggregation platform 102.

FIG. 2B is a schematic block diagram of a system and process flow for accessing the cloud-based database 202 described in FIG. 2A. The data aggregation platform 102 first authenticates and retrieves tokens from a user pool 230. The data aggregation platform 102 then exchanges tokens for database credentials with the identity pool 232. The data aggregation platform 102 is then granted access to the could-based database 202 based upon the credentials.

The user pool 230 is a user directory associated with the cloud-based database 202. With the user pool 230, users can sign into the data aggregation platform 102 through a mobile application, computer-based application, web-based user interface, third-party identity provider, and so forth. Whether users sign in directly or through a third party, all members of the user pool 230 have a director profile that can be accessed. The user pool 230 enables sign-up and sign-in services for the data aggregation platform 102 and further enables social sign-in with outside services, including outside social media networks. The user pool 230 stores a directory, and this directory may be managed, and permissions may be assigned to users within the director.

The identity pool 232 creates temporary credentials to access the cloud-based database 202. The identity pool 232 supports anonymous guest users and social sign-in through outside parties, including third-party social media network.

The system 200 authenticates users by leveraging the user pool 230. After a successful sign-in through the user pool 230, the data aggregation platform 102 creates user pool groups to manage permissions and to represent different types of users. The data aggregation platform 102 creates user groups defined by a type of data permission for that group.

The data aggregation platform 102 may access the cloud-based database 202 through an Application Program Interface (API) Gateway. The API Gateway validates the tokens from a successful user pool 230 authentication and uses those tokens to grant users access to the resources within the data aggregation platform 102 and the cloud-based database 202. The data aggregation platform 102 leverages the user groups defined within the user pool 230 to control permissions with the API Gateway by mapping group membership to roles within the user pool 230. The user groups that a user is a member of are included in the identification token provided by a user pool 230 when the user signs into the data aggregation platform 102. The data aggregation platform 102 submits the user pool tokens with a request to the API Gateway for verification by an authorizer for the cloud-based database 202.

In an embodiment, a unique user pool 230 is created for each tenant within the data aggregation platform 102. This approach provides maximum isolation for each tenant and allows the data aggregation platform 102 to implement different configurations for each tenant. Tenant isolation by user pool 230 allows flexibility in user-to-tenant mapping and allows multiple profiles for the same user. Additionally, in this implementation, a unique hosted user interface may be assigned to each tenant independently, and the data aggregation platform 102 will automatically redirect each tenant to their tenant-specific user interface instance.

In an embodiment, a single user may be mapped to multiple tenants without recreating the user's profile within the data aggregation platform 102. In this embodiment, a data package client is executed for each tenant, and this data package client enables the tenant external IdP as the only allowed provider for that data package client. Data package client-based multi-tenancy requires additional considerations for username, password, and more to authenticate users with the native accounts. When the hosted user interface is in use, a session cookie is created to maintain the session for the authenticated user. The session cookie also provides SSO between data package clients in the same user pool 230.

In an embodiment, the data aggregation platform 102 implements role-based access control. The identity pools 232 assign authenticated users a set of temporary, limited-privilege credentials to access the resources in the cloud-based database 202. The permissions for each user are controlled through roles created within the data aggregation platform 102. The data aggregation platform 102 defines rules to choose the role for each user based on claims in the user's identification token. The rules enable the data aggregation platform 102 to map claims from an identity provider token to a role. Each rule specifies a token claim (such as a user attribute in the identification token from the user pool 230), match type, a value, and a role. The match type can be Equals, NotEqual, StartsWith, or Contains. If a user has a matching value for the claim, the user can assume that role when the user gets credentials. For example, the data aggregation platform 102 may create a rule that assigns a specific role for the users with a custom: dept custom attribute value of Sales.

Rules are evaluated in order, and the role for the first matching rule is used, unless a custom role is specified to override the order. The data aggregation platform 102 may set multiple rules for an authentication provider in the identity pool 232. Rules are applied in order. The order of the rules may be altered. The first matching rule takes precedence. If the match type is NotEqual, and the claim does not exist, then the rule is not evaluated. If no rules match, the role resolution setting is applied to either use the default authenticated role or to deny. The data aggregation platform 102 specifies a role within the API connection to the cloud-based database 202 to be assigned when no rules match in the ambiguous role resolution process. For each user pool 230 or other authentication provider configured for an identity pool 232, the data aggregation platform 102 may assign numerous rules.

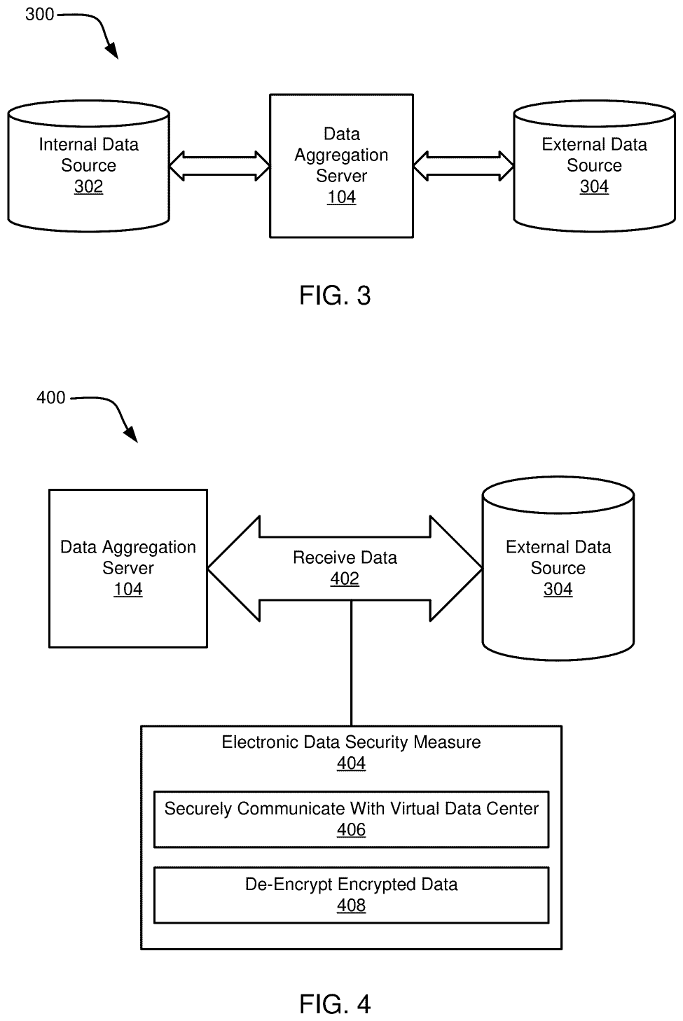

FIG. 3 is a schematic diagram of a system 300 for data communication between a data aggregation server 104 and internal and external data sources. The data aggregation server 104 identifies and quantifies the availability and cost of items based on outside data. Specifically, the data aggregation server 104 may receive information regarding, for example, items available on the market for purchase, the pricing of items, items currently available at a facility, the status of items held by a facility, the profit margin for billing the use of items to a patient, the efficacy of certain items, third-party reviews, and opinions about certain items, and so forth. The data aggregation server 104 may communicate with one or more of an internal data source 302 and an external data source 304.

In an embodiment, the data aggregation server 104 communicates directly with an external data source 304 that is managed or owned by a third-party entity. The data aggregation server 104 may communicate by way of SSL-encrypted HTTP connections. In an embodiment, the external data source 304 is a relational database, and the data aggregation server 104 communicates with the relational database by way of an Application Program Interface (API). In an embodiment, the external data source 304 is an encrypted hard drive that has been shared with the data aggregation server 104. In an embodiment, the external data source 304 is a virtual data center, and the data aggregation server 104 access the data on a virtual server after signing in or undergoing some other authentication step.

In an embodiment, the data aggregation server 104 communicates with an internal data source 302 that is not managed by some other third-party entity. The internal data source 302 may include a file that has been downloaded or otherwise received from some third-party entity. After the file has been downloaded, the file can be managed and manipulated by the data aggregation server 104. The internal data source 302 may include an encrypted hard drive that is provided by a third-party.

In an embodiment, the data stored in the internal data source 302 has been “cleaned” or pared down to only include necessary or critical information. This can be beneficial to ensure the totality of the data is a usable size that can be efficiently queried, analyzed, and manipulated. For example, the raw data retrieved from the external data source 304 may include numerous data fields that are not necessary for generating and maintaining aggregated data forms as described herein. The unnecessary data may be eliminated, and only the necessary data may be stored on the internal data source 302. In an embodiment, the raw data is cleaned and stored in a relationship database.

The data aggregation server 104, or some other module in communication with the data aggregation server 104, may create intermediary files or tables within a relational database. The intermediary files or tables may include certain information columns that are pertinent to answer a specific question, such as whether an item is owned by a certain facility. This can be beneficial to ensure that each intermediary file or table is no bigger than it needs to be to include all necessary information for answering the specific question. This decreases the amount of disc storage and/or Random-Access Memory (RAM) needed to analyze the information and calculate the answer to the specific question.

FIG. 4 is a schematic diagram of a system 400 for performing electronic data security measures on data received from the external data source 304. The data aggregation server 104 receives claims data (see 402) from an external data source 304. In some cases, the data may be private or encrypted, such as item-use data for procedures that were performed in the past. This data may be received as part of a healthcare claim and may include private or personal information.

In an embodiment, the data aggregation server 104 may receive data by securely communicating with a virtual data center. The virtual data center may be provided by a private or public healthcare entity. In an embodiment, an account may be created for a user associated with the data aggregation server 104, and the user could sign into the virtual data center with the account. The user could then access the data stored in the virtual data center by way of the account. The data may be encrypted or non-encrypted based on the security measures of the virtual data center. In an embodiment, the data may be non-encrypted when viewed by way of a network connection, and the data may be encrypted if downloaded for offline use and manipulation. If the data is downloaded in an encrypted form, then the data must be de-encrypted prior to analysis and manipulation.

In an embodiment, the data aggregation server 104 receives data by way of an encrypted hard drive. The encrypted hard drive may be provided by the source of the data, such as private or public healthcare entity. In an embodiment, the data aggregation server 104 receives claims data by way of an encrypted file that has been downloaded by way of a network connection. The data aggregation server 104 undergoes an electronic data security measure 404 by de-encrypting the claims data.

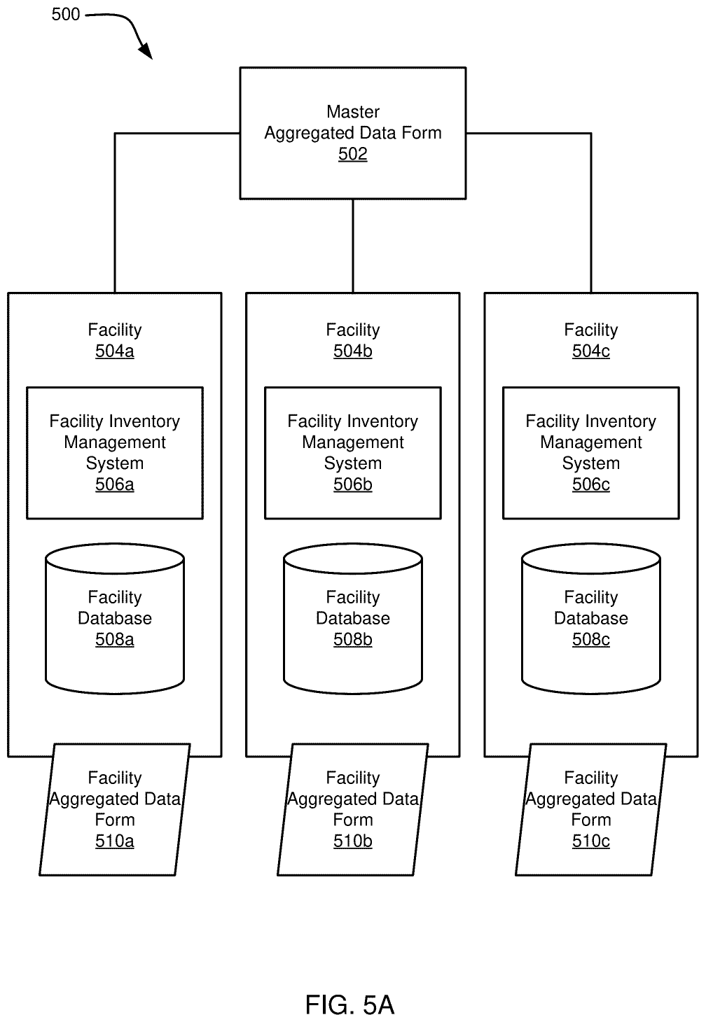

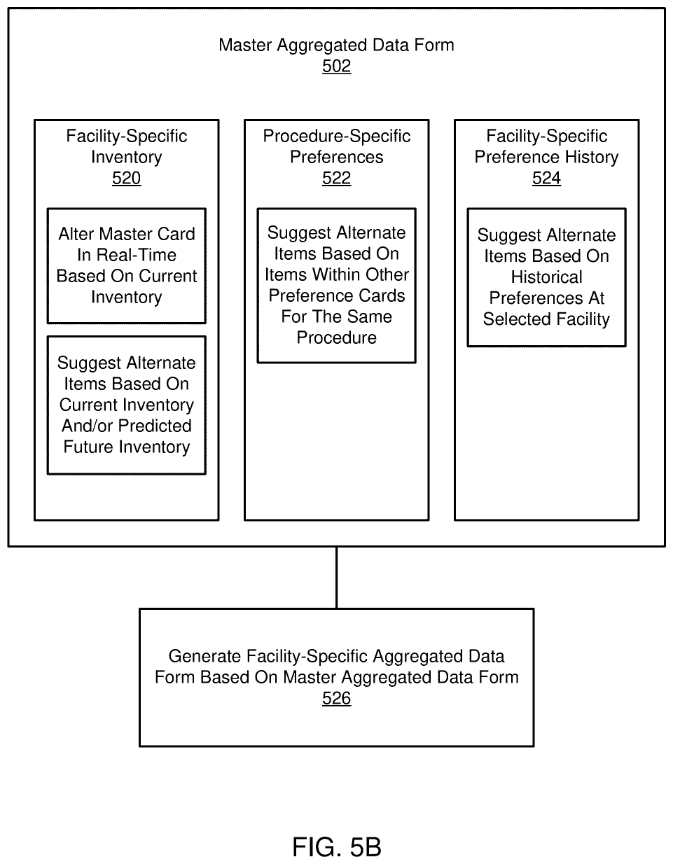

FIG. 5A is a schematic block diagram of a system 500 and method for applying a master aggregated data form 502 to one or more facilities 504a, 504b, 504c (may be generically referred to herein as facility 504). The data aggregation server 104 communicates with facility inventory management systems 506 (see 506a, 506b, 506c) associated with different facilities 504. The data aggregation server 104 additionally communicates with one or more facility databases 508 (see 508a, 508b, 508c) associated with those facilities 504. The data aggregation server 104 suggests edits to the master aggregated data form 502 based on the inventory data and facility data received from the facilities 504. These suggested edits may be reflected in the facility aggregated data forms 510 (see 510a, 510b, 510c) for each of the different facilities 504. As discussed herein, the “facility aggregated data forms” 510 may alternatively be referred to as “slave aggregated data forms” to refer to the master-slave data architecture.

Traditionally, the healthcare industry implements the HL7 standard system, and this system does not have any specifications for the management of aggregated data form data. The data aggregation server 104 may communicate with existing systems by way of custom-built APIs to retrieve real-time data from the existing systems. The data aggregation server 104 may retrieve inventory lists through the HL7 interface. The aggregated data form system 104 analyzes changes made against a facility card and pass it up to the master card for evaluation by the practitioner, since the request may have happened at the practitioner's request, and this would allow them to apply the change more broadly than just at the one facility.

FIG. 5B is a schematic block diagram of process flow 600 for generating and suggesting procedure-specific and/or facility-specific aggregated data form items based on a master aggregated data form and additional data sources. The process flow 600 illustrated in FIG. 5B may be performed by an AI/ML engine, wherein the AI/ML engine is trained on datasets comprising aggregated data forms for healthcare practitioners across numerous facilities and geographical areas, along with real-time data applicable to items that may be used in aggregated data forms.

The master aggregated data form 502 is generated by a practitioner. The data aggregation server 104 analyzes the items selected in the aggregated data form, along with the procedure associated with the aggregated data form, and the facilities where the procedure will be performed, and provides one or more suggestions for amending the master aggregated data form 502. The data aggregation server 104 analyzes facility-specific inventory 520, which may include altering the master card in real-time (or suggesting amendments) based on current inventory of items at a facility where the procedure will be performed. This may additionally include suggesting alternate items based on current inventory and/or predicted future inventory.

The data aggregation server 104 analyzes procedure-specific preferences along with the item medical device database 112 and may provide suggestions on alternate items for the applicable procedure. The data aggregation server 104 may receive information from other practitioners performing the same procedure, reviews written about items in the item catalog, and information in the item catalog indicating that certain items can be used for the applicable procedure. The data aggregation server 104 suggests to the practitioner that alternate items may be used in the applicable procedure. In an implementation, the practitioner may request suggested items for an applicable procedure, and the data aggregation server 104 determines the suggested items based on other aggregated data forms within the system for the same procedure and/or publicly available aggregated data forms for the applicable procedure.

The data aggregation server 104 may additionally analyze facility specific preference history 524 and suggest alternate items based on the practitioner's historical preferences at a selected facility. The practitioner and/or the facility may manually input the practitioner's preferences at that facility. The data aggregation server 104 may identify these preferences based on data indicating what products the practitioner used when performing procedures at the facility.

The data aggregation server 104 analyzes items on other aggregated data forms that are used for similar or identical procedures. The data aggregation server 104 determines whether the aggregated data form includes specific items that require other items to be present to be used. This is the case when, for example, there is a disposable piece to be used in operating a piece of machinery, or in the case of a supply that is necessary to the use of the equipment, such as jelly for the use of an ultrasound. The data aggregation server 104 references outside data, such as the manufacturers'recommendations, studies on outcomes of the use of different items, and what other practitioners in the field are using based on their own aggregated data forms.

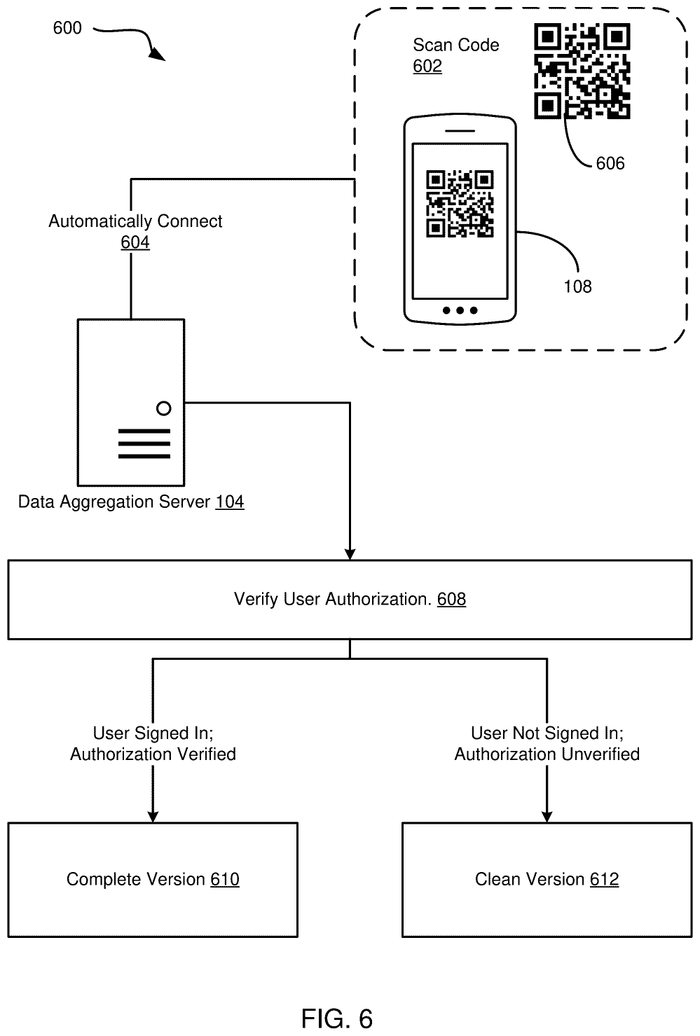

FIG. 6 is a schematic block diagram of a system and process flow 600 for utilizing a scannable code to redirect to a virtual file representing an aggregated data form. The process flow 600 is performed by the data aggregation server 104 and a personal device 108, such as a mobile phone with a camera.

The process flow 600 is initiated by scanning a scannable code 606 at 602. The code scanning 602 may be performed by a personal device 108 comprising a camera or other scanner for scanning the scannable code 606. The scannable code 606 may specifically include a quick response (QR) code or other suitable code. The scannable code 606 is associated with a certain aggregated data form, and may be included on a virtual file or hardcopy file associated with the aggregated data form.

The process flow 600 includes automatically connecting 604 the personal device 108 with the data aggregation server 104. The data aggregation server verifies user authorization at 608 to determine whether the user is authorized to view all information associated with the aggregated data form. If the user is signed in to the data aggregation platform 102 and the user's authorization to view the aggregated data form is verified, then the data aggregation server 104 enables the user to view the complete version 610 comprising all information associated with the aggregated data form. The complete version 610 may or may not include editing authorization, dependent upon the user's access permissions. If the user is not signed in to the data aggregation platform 102 and the user's authorization to view the aggregated data form is unverified, then the data aggregation server 104 will only permit the user to view a clean version 612 of the aggregated data form, which includes some information but removes any personally identifying information or other sensitive information. The clean version 612 may specifically include any information that may be considered a violation of HIPPA (Health Insurance Portability and Accountability Act).

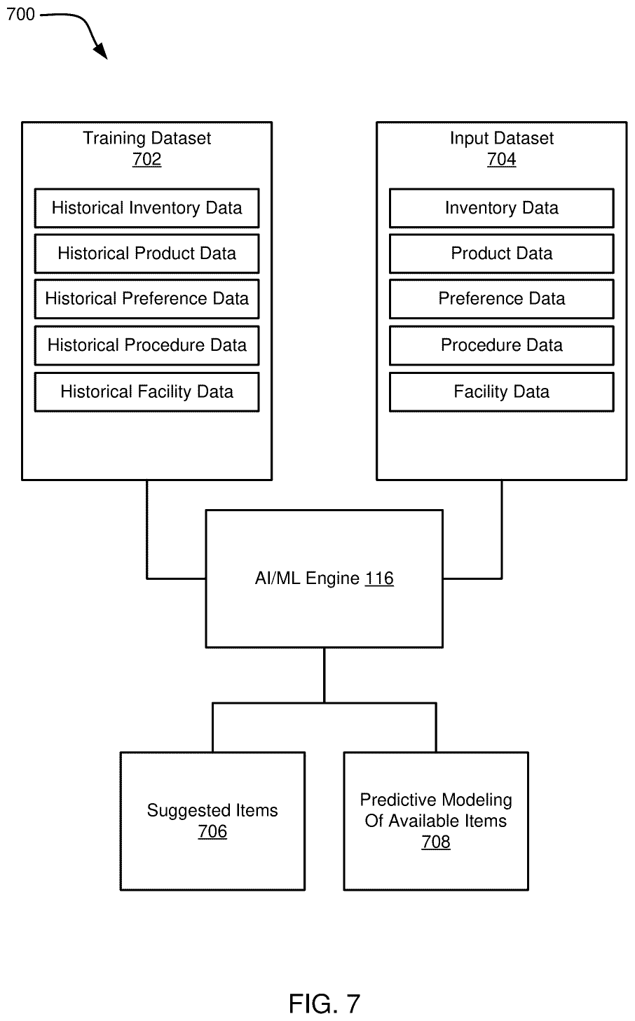

FIG. 7 is a schematic block diagram of a dataflow 700 for training an AI/ML engine, providing input data to the AI/ML engine, and receiving an output calculation from the AI/ML engine. The AI/ML engines described herein may be trained based on a training dataset 702 including one or more of historical inventory data, historical product data, historical preference data, historical procedure data, and historical facility data. When the AI/ML engine is trained, the AI/ML engine may be configured to perform real-time analysis on data within an input dataset 704, including one or more of inventory data, product data, preference data, procedure data, and facility data. These datasets are fed to the AI/ML engine 116. The AI/ML engine 116 outputs one or more of suggested items 706 of an aggregated data form and/or predictive modeling of available items 708 based on the input data.

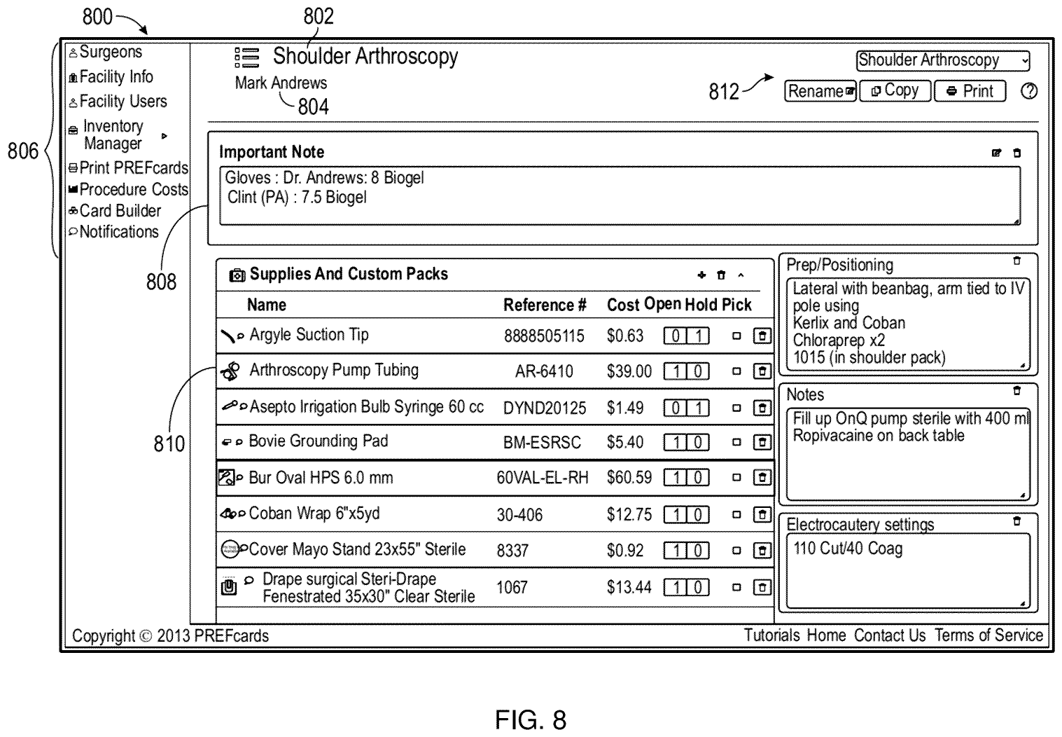

FIG. 8 is a screenshot of an example user interface 800 for entering item selections for an aggregated data form. The screenshot indicates that a listing of available items may be presented to the user, and the user may select any quantity of the listed items as applicable for the procedure. The listed items may be pulled in real-time from one or more of an inventory management solution 110 and/or the item medical device database 112.

In the example illustrated in FIG. 8, the user interface 800 is rendered to illustrate components of a surgical preference card, and further to enable a user to edit the surgical preference card. In this example implementation, the user interface 800 includes a procedure title 802, and in the example user interface 800 illustrated in FIG. 8, the aggregated data form is associated with a shoulder arthroscopy. The user interface 800 includes a practitioner name 804, and in this case, the practitioner (surgeon) is Mark Andrews.

The user interface 800 includes an accessible menu 806 comprising a plurality of options, including, for example, “Surgeons,” “Facility Info,” “Facility Users,” “Inventory Manager,” “Print Preference Cards,” “Procedure Costs,” “Card Builder,” and “Notifications.” Each of the items within the accessible menu 806 is an interactive button that may be selected by a user. When a user selects the interactive button, the user interface 800 will redirect and enable the user to view and/or amend additional information relating to the selected menu item. The accessible menu 806 may remain accessible at one portion on the user interface 800 while the user interacts with other portions of the user interface 800. For example, the accessible menu 806 may be “locked” and remain accessible at the left-hand sidebar (as shown in FIG. 8), on the right-hand sidebar, on the top of the user interface 800, and/or on the bottom of the user interface. The accessible menu 806 may remain visible and accessible even when the user scrolls the user interface 800.

The user interface 800 includes an input box 808. In the implementation illustrated in FIG. 8, the input box 808 displays an “Important Note” regarding the preference card, and specifically indicates the name of the surgeon, the gloves preferred by the surgeon, the name of the physician assistant, and the gloves preferred by the physician assistant. It should be appreciated that the input box 808 may display any suitable information depending on the implementation. The user may interact with the input box 808 to amend the information that is displayed therein.

The user interface 800 includes a dynamic list 810. The user interface 800 may include a plurality of different dynamic lists 810 each associated with a different topic. In the implementation illustrated in FIG. 8, the dynamic list 810 includes a listing of products under the grouping of “Supplies And Custom Packs.” The product listing is dynamic and editable such that a user may quickly alter the items within the product listing, alter the quantity of each item within the product listing, delete items from the product listing, and see real-time up-to-date information regarding each item within the product listing. The dynamic list 810 illustrated in FIG. 8 includes, as an example, “Argyle Suction Tip,” “Arthroscopy Pump Tubing,” “Asepto Irrigation Bulb Syring 60 cc,” “Bovie Grounding Pad,” “Bur Oval HPS 6.0 mm,” “Coban Wrap 6″×5 yd,” “Cover Mayo Stand 23×55″ Sterile,” and “Drape Surgical Steri-Drape Fenestrated 35×30″ Clear Sterile.” It should be appreciated that the dynamic list 810 may include a listing of any suitable items, including, for example, products, names of people, names of places, appointments, tasks to be performed, and so forth.

In an embodiment, the dynamic list 810 is automatically updated in real-time based on information retrieved by the data aggregation server 104, including, for example, data from the web scraping 140, data from the invoicing and disbursement tracking database 116, global data 114, item medical device database 112 data, and data from the inventory management solution 110. The data aggregation server 104 may automatically update the dynamic list 810 to indicate, for example, that the SKU for an item has changed, the pricing for an item has changed, the quantity available of an item has changed, and so forth.

The user interface 800 includes update input boxes 812 wherein a user may amend or update information displayed on the user interface 800. The update input boxes 812 may include, for example, a button enabling a user to change the name of a procedure, surgeon, item within the dynamic list 810, and so forth. The user interface 800 further enables a user to print, copy, or save information illustrated in the user interface 800.

The user interface 800 may flag certain items within the dynamic list 810. In the example illustrated in FIG. 8, the shaded grey portion for “Bur Oval HPS 6.0 mm” indicates that the item has a cost higher than a threshold configurable by the facility. This enables the facility to easily identify items that are the largest contributors to the cost of the procedure.

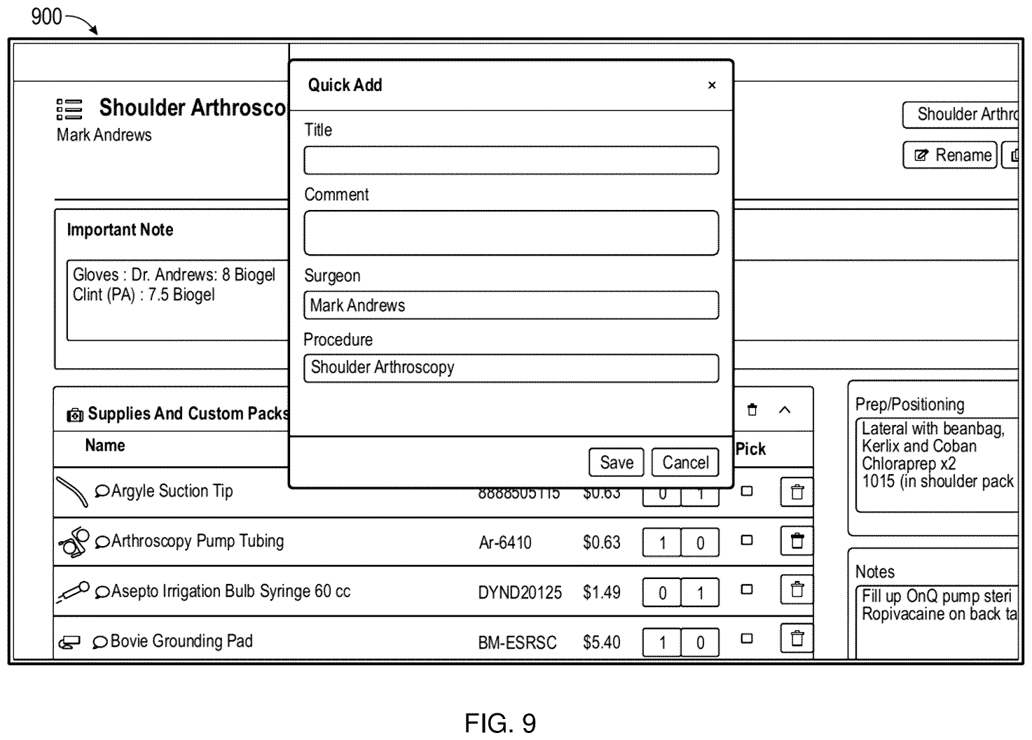

FIG. 9 is a screenshot of an example user interface 900 for quickly adding item selections to an aggregated data form. The data aggregation platform 102 enables a user to quickly add a note or item to an existing aggregated data form. The data aggregation platform 102 records the comment and may provide a form of knowledge capture. The user may later review the comments and make changes, then mark the notification or comment as being resolved.

The data aggregation server 104 may implement machine learning and tagging to extract the actions that need to be taken and present those actions as options for the user to accept as changes to the aggregated data form. This may be done rather than having the user evaluate the requested change. This recognition includes identifying the practitioner and procedure(s) from the freeform text of the comment.

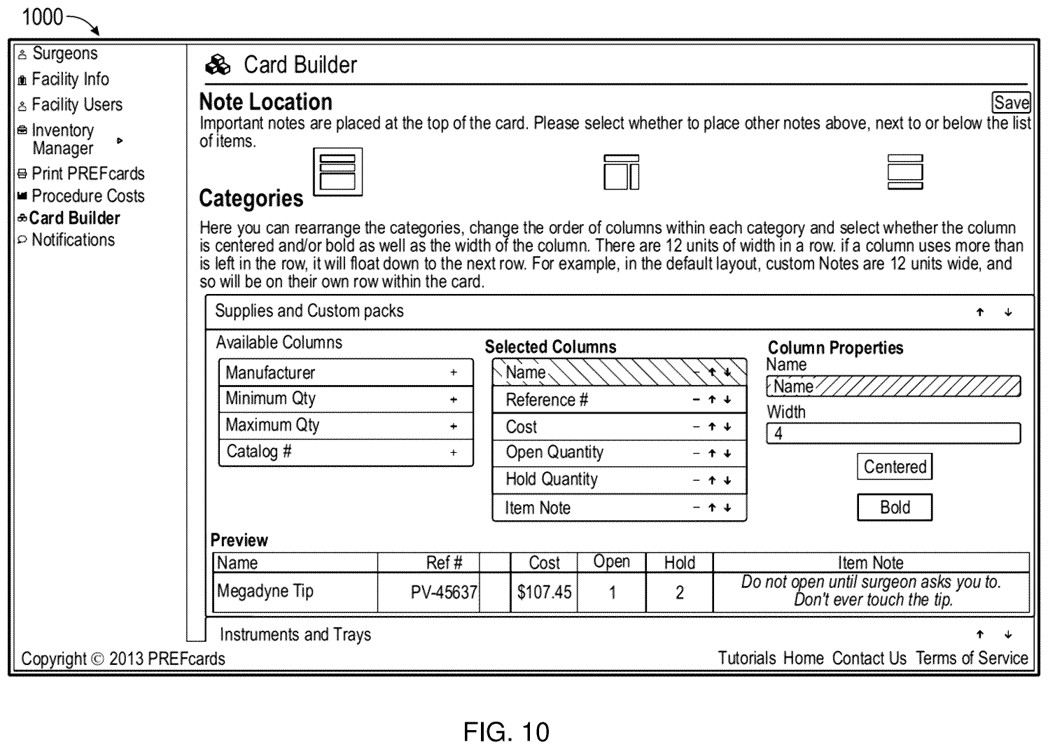

FIG. 10 is a screenshot of an example user interface 1000 for building an aggregated data form. The data aggregation platform 102 enables users to specify the organization and layout of the aggregated data form along with the number of columns of information on the aggregated data form, and what data should be associated with those columns of information. A facility may request a certain layout for aggregated data forms at that facility, and the requested layout will be propagated to all cards that that facility. This ensures the cards can be easily read and digested by facility personnel when gathering items for a procedure. The layout for an aggregated data form may be different on the practitioner's end versus the facility's end. Different accounts within one facility may request different layouts for the aggregated data forms used at that facility.

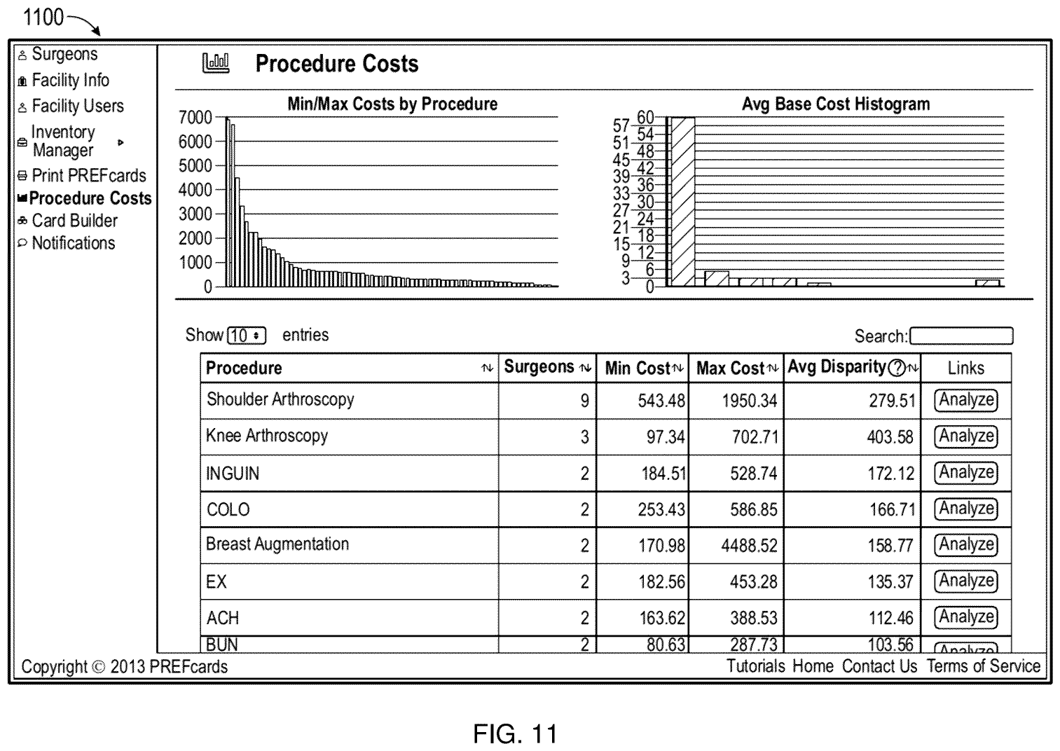

FIG. 11 is a screenshot of an example user interface 1100 for calculating predicted procedure costs based on items selected on an aggregated data form. The data aggregation platform 102 calculates the minimum and maximum predicted cost for performing a procedure based at least in part on the items selected in the associated aggregated data form. The predicted cost may be based on real-time data received from invoicing and disbursement tracking 116, global data 114, and or item medical device database 112 as discussed herein.

The minimum cost is the sum of the costs of all items on the aggregated data form which are marked as open, indicating that the user preparing for the procedure should have the items open and ready to go for the practitioner. If the item is opened, it cannot be placed back into the stock room to be used on a different procedure. The maximum cost is calculated by adding up the costs of all open and hold items, which reflects 100% utilization of the items requested to be pulled for the procedure. This is not truly a maximum cost, as anything could happen during a procedure that could require additional items that were not on the original card but having a large difference between the minimum and maximum costs is usually an indicator that the practitioner is preparing for additional items that may need to be used but is being prudent in the number of those items that they open before the case starts. FIG. 11 presents all procedures across all practitioners at the facility. The table groups the practitioners by procedure and calculates the minimum cost across all the practitioners'minimum costs. The maximum cost in the table is the maximum across all practitioners'maximum costs.

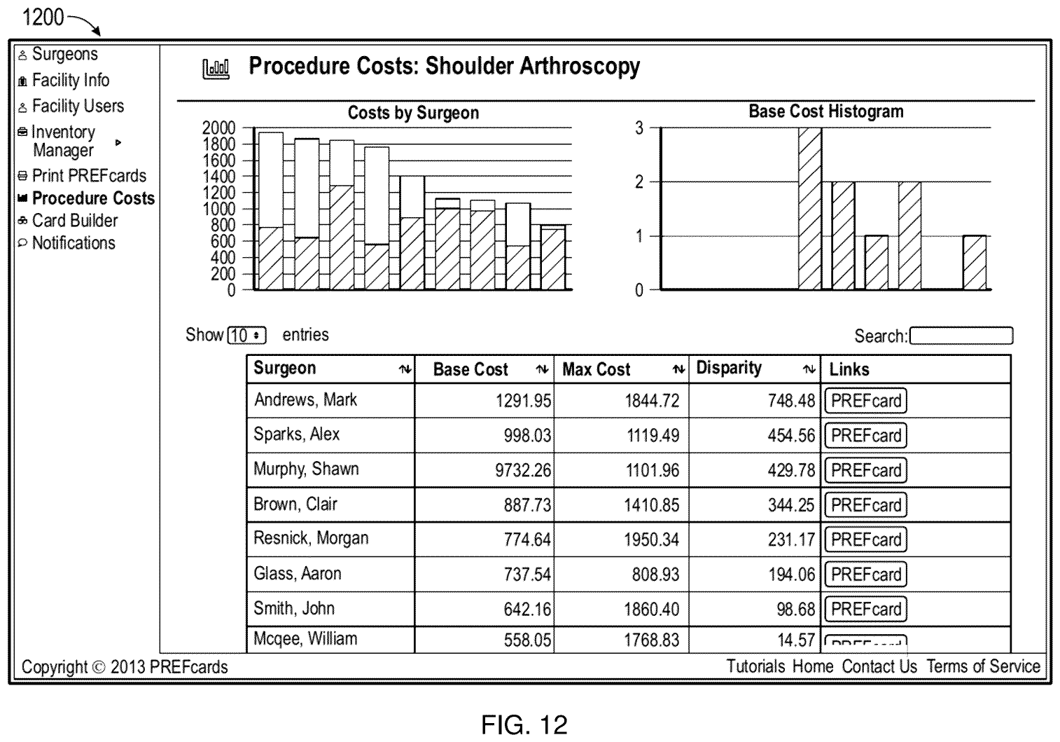

FIG. 12 is a screenshot of an example user interface 1200 for providing cost-variation data for a certain procedure. The data aggregation platform 102 may provide cost analysis per-practitioner at a certain facility, within a healthcare network, within a healthcare system, within a geographic region, and so forth. The cost analysis data may be scrubbed such that all cost information is anonymous and does not reveal the practitioner or facility where the procedure was performed. The cost analysis may indicate the name of the practitioner and/or the facility where the procedure was performed (as illustrated in FIG. 12). The cost analysis may be based on the items selected in the aggregated data forms for each of the listed practitioners. The cost analysis may be broken down for each procedure and/or for each practitioner across all procedures performed by that practitioner.

FIG. 12 reflects a drill-down view of FIG. 11, wherein a single row of data from FIG. 11 is illustrated for an individual practitioner/procedure combination that make up that row's constituent members. The screenshot illustrates in the charts whether there are any outliers in the data, as well as the magnitude of variance in the table below.

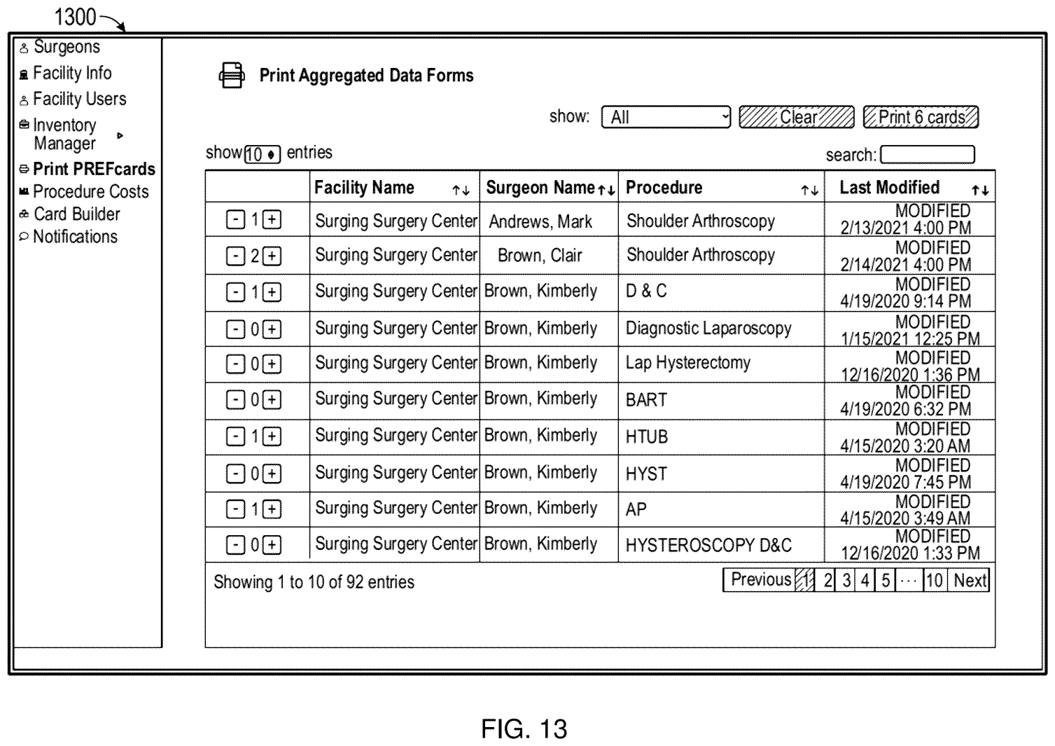

FIG. 13 is a screenshot of an example user interface 1300 for selecting, printing, viewing, and communicating aggregated data forms. The print page provides a plurality of aggregated data forms that may be viewed by the user associated with the account. The data aggregation server 104 may communicate with a facility and/or practitioner to identify which procedures are scheduled to be performed in an upcoming time period, and the data aggregation platform 104 may provide the applicable aggregated data forms that will need to be referenced during the upcoming time period. In an implementation, the data aggregation server 104 automatically pulls all aggregated data forms for procedures schedule to be performed the following day and provides those preferences cards to the applicable persons at the facility where the procedures will be performed.

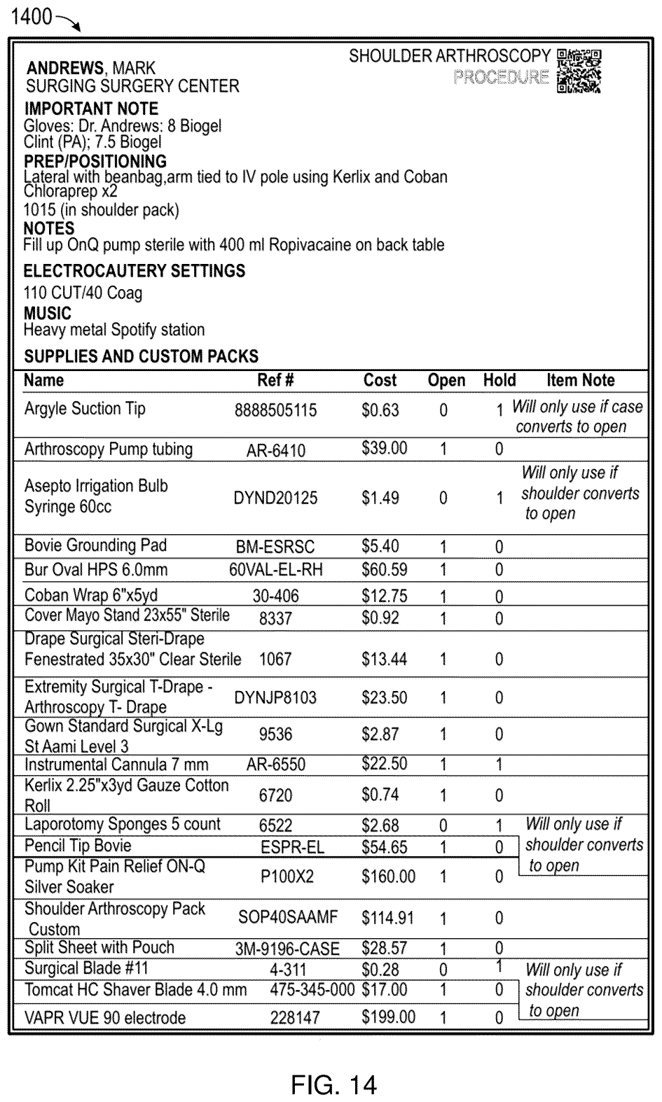

FIG. 14 is a screenshot of an applicable user interface and virtual file 1400 output by the data aggregation server 104. The example screenshot illustrates a document comprising an aggregated data form for a shoulder arthroscopy procedure to be performed by ANDREWS, MARK. The aggregated data form includes a QR code that may be scanned by a system at the facility. The QR may provide instructions to an inventory management solution 110 that indicates which items should be selected and retrieved for the procedure. The QR may include computer-based instructions indicating that a robotic component of the inventory management solution 110 should retrieve the items listed on the aggregated data form.

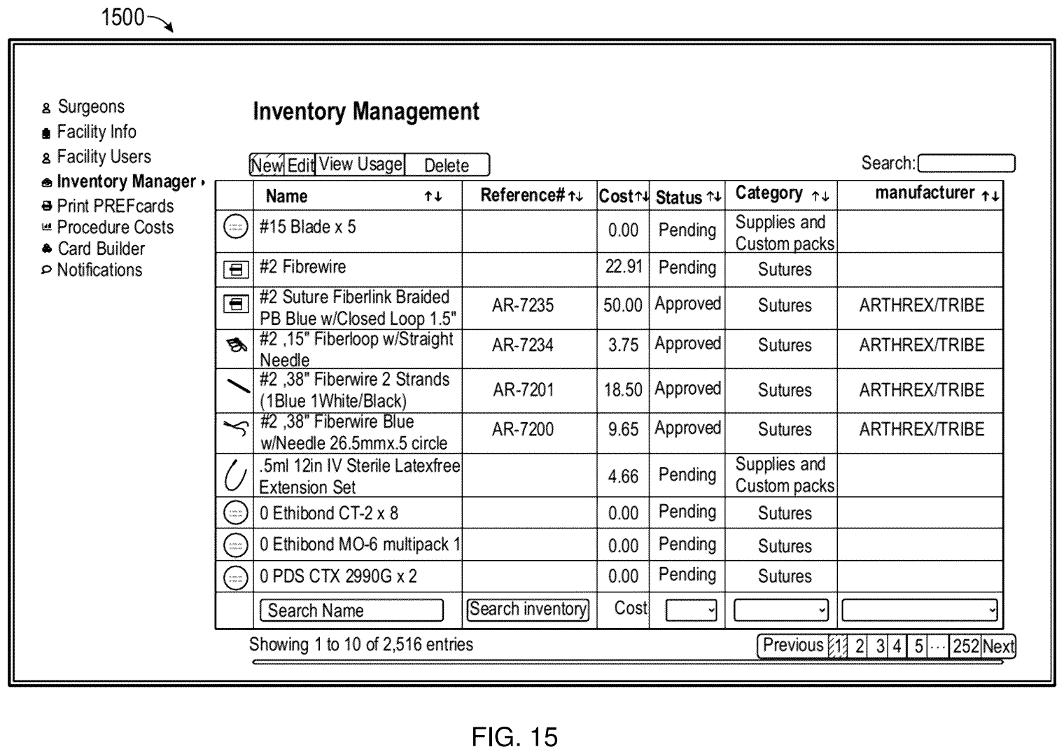

FIG. 15 is a screenshot of an example user interface 1500 of the data aggregation platform 102. The screenshot illustrates inventory management data indicating the names, reference identifiers, cost, status, category, and manufacturer for certain items. The items listed in the screenshot may be items used by a certain practitioner or facility, items owned by a facility, items that need to be purchased by a facility, items that will be used in upcoming scheduled procedures, and so forth. The list of items is hosted on the data aggregation server 104 and may additionally include items populated from an internal or external connection to software systems run by a facility inventory management system or item catalog.

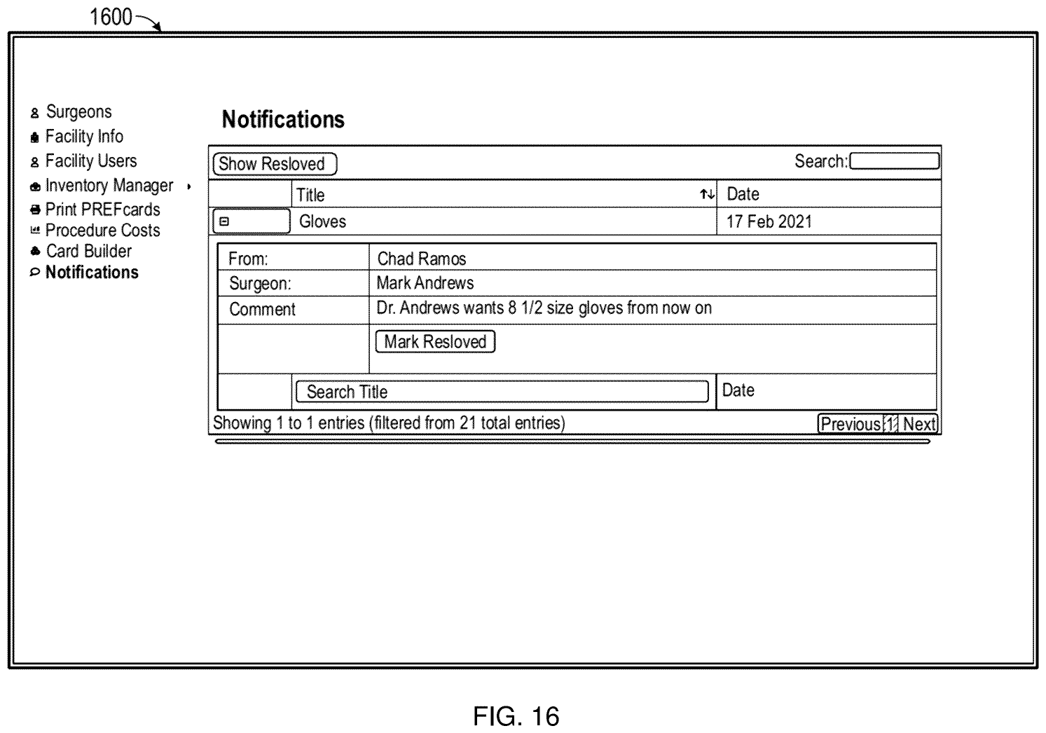

FIG. 16 is a screenshot of an example user interface 1600 of the data aggregation platform 102. The screenshot indicates notifications that may be generated by a practitioner and/or facility and transmitted to other accounts. In the example notification, a practitioner (Mark Andrews) is requesting a certain glove size for all procedures moving forward. The component illustrated in FIG. 16 allows for review of comments captured in the knowledge capture interface illustrated in FIG. 9. These comments can then be marked as resolved once the required changes are made to the cards indicated.

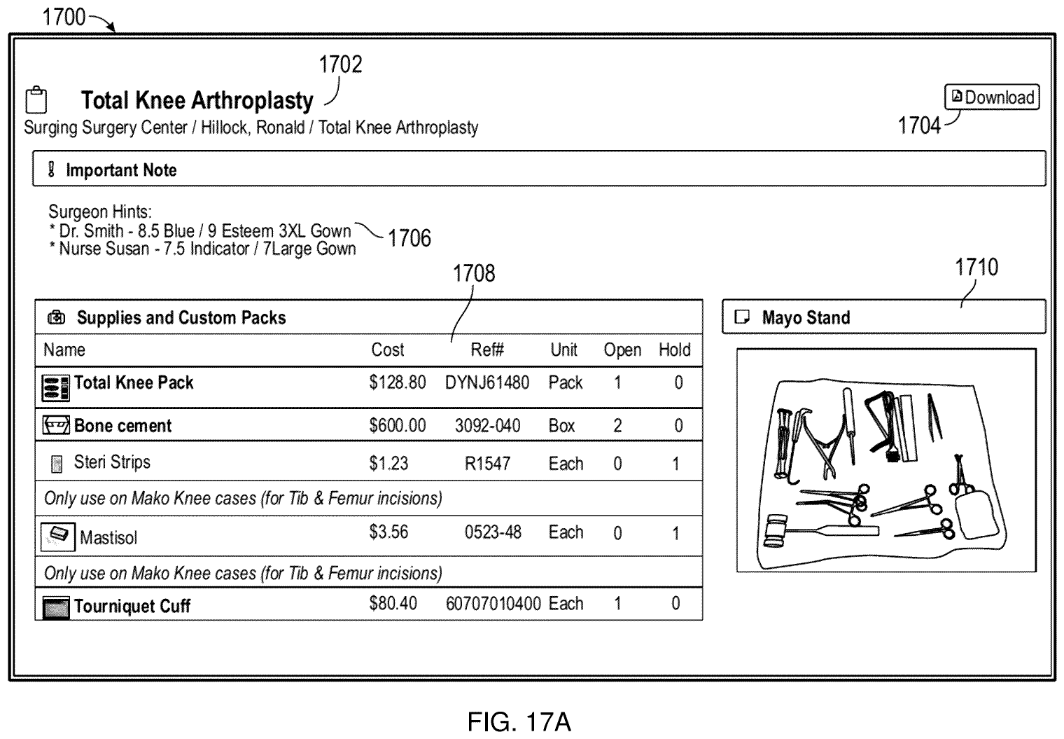

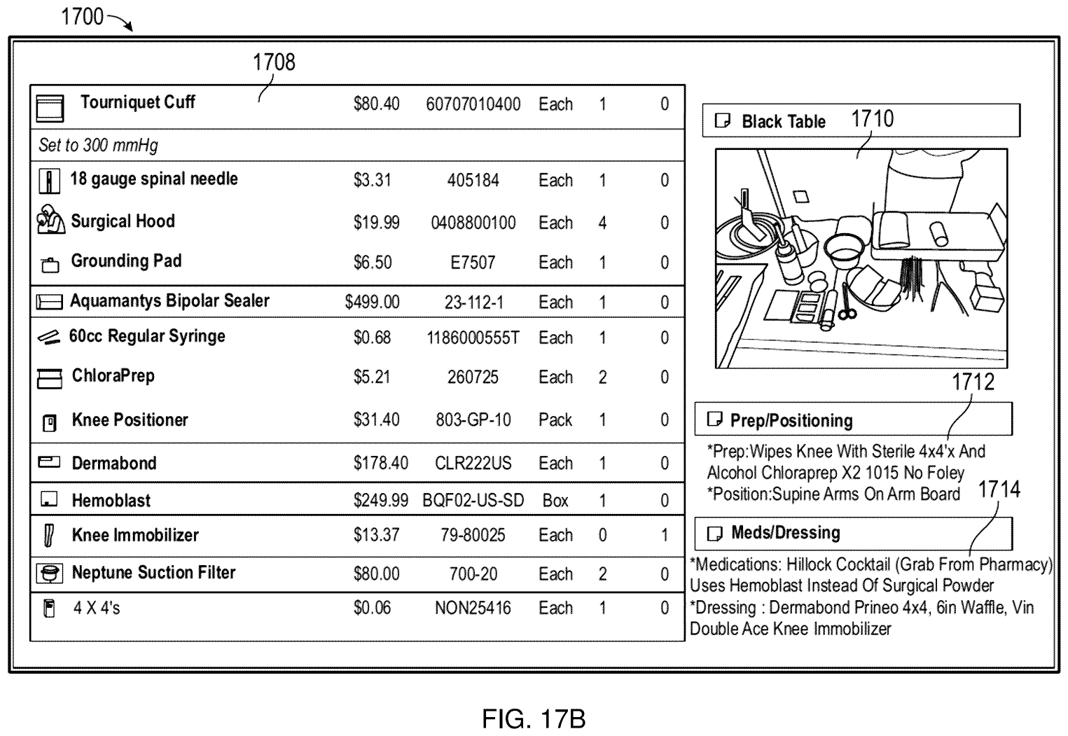

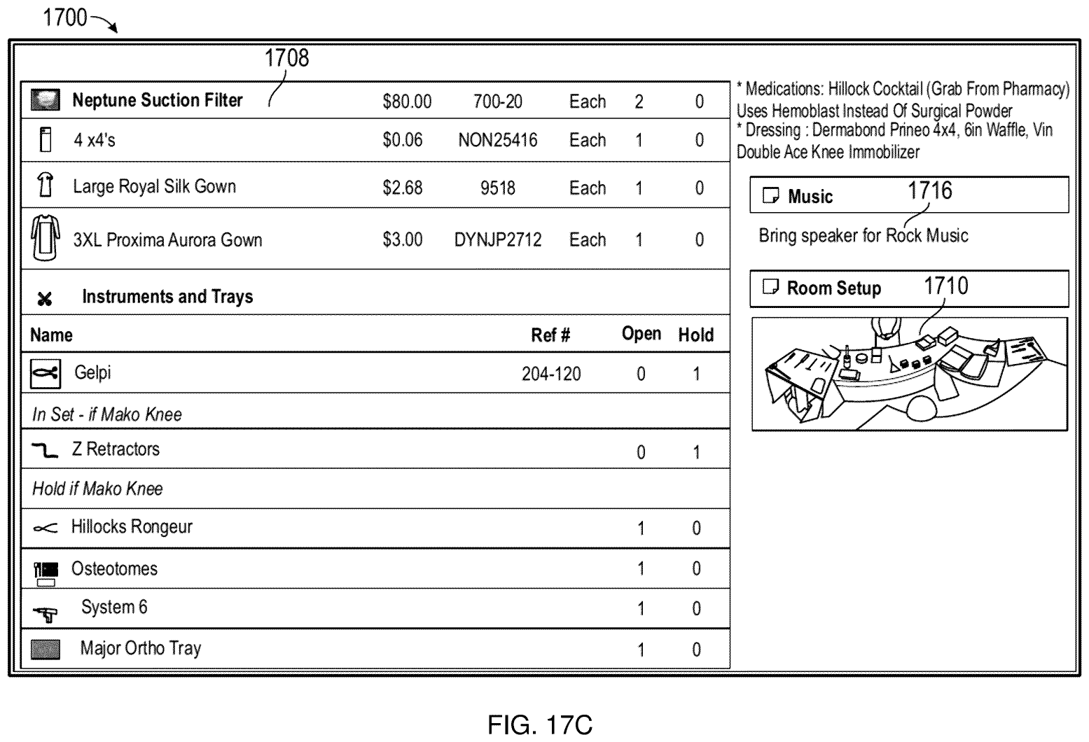

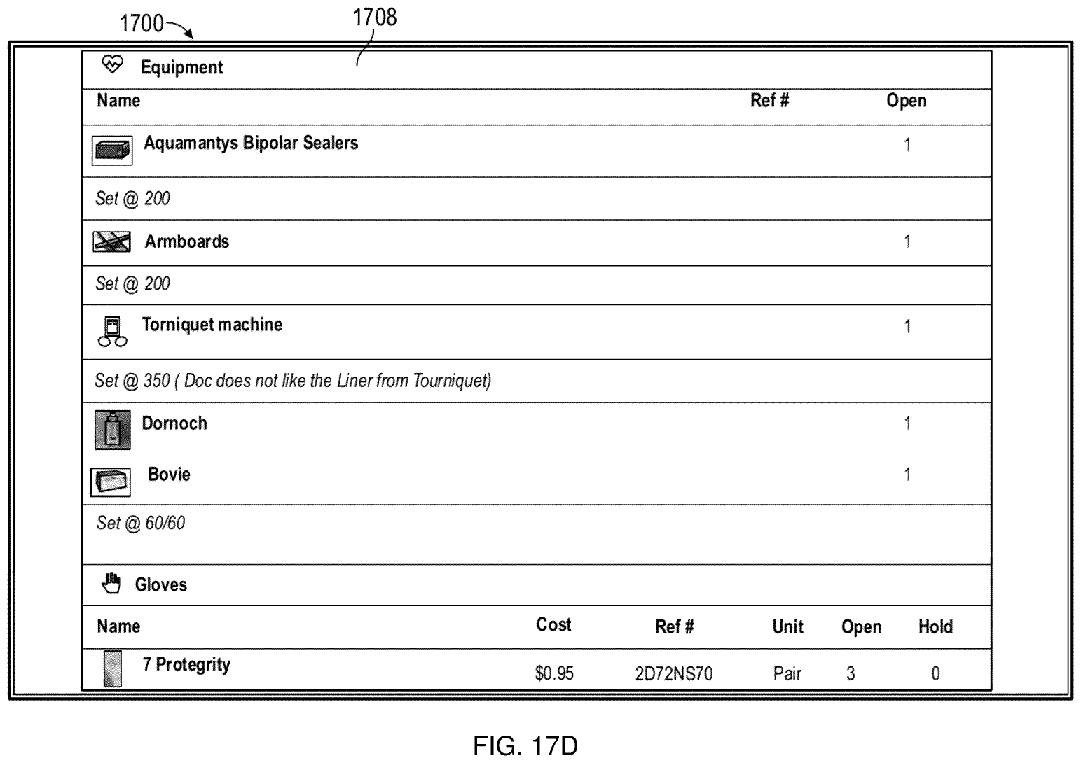

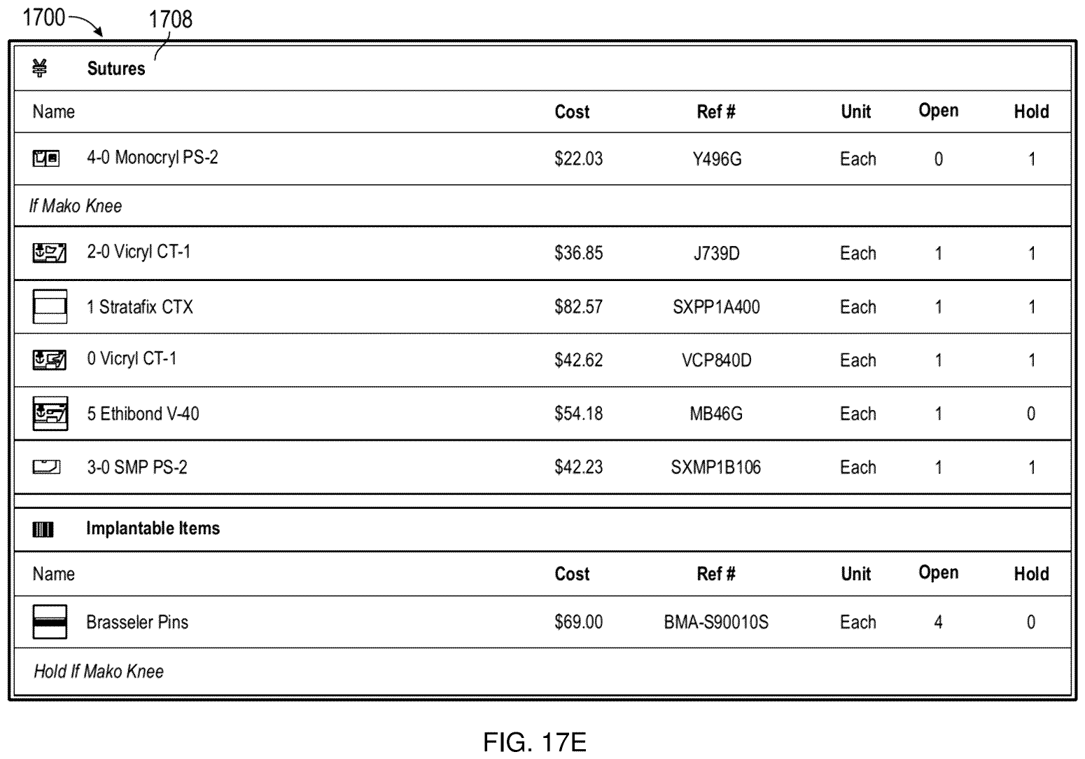

FIGS. 17A-17E illustrate screenshots of an example user interface 1700 for presenting an aggregated data form. The screenshots illustrated in FIG. 17A-17E capture the example aggregated data form in order from top to bottom. The organization and layout of the user interface 1700 may be altered based on the preference of the healthcare administrator or provider, surgeon, surgical staff, and so forth.

The user interface 1700 includes a title of the surgery. In this case, the title indicates that the aggregated data form includes instructions for a total knee arthroplasty. The aggregated data form is intended for Surging Surgery Center with surgeon Ronald Hillock. The user interface 1700 includes a means for downloading 1704 the aggregated data form. The downloaded aggregated data form may be converted to a desired file format, such as a PDF, word document, plain text, and so forth. The user interface 1700 includes a second for providing important notes 1706 about the surgery. The important notes 1706 may be provided by the surgeon, surgical center, healthcare administrator, and so forth. The important notes 1706 may be surgery-specific, surgeon-specific, patient-specific, surgical center-specific, and so forth. The important notes 1706 may recite different information for different surgeries performed by the same surgeon at the same surgical center. The user interface 1700 includes a listing of supplies 1708. The supplies 1708 may include listing of custom packs of supplies as illustrated in FIG. 17A. The user interface 1700 includes surgical setup images 1710 for illustrating how the surgeon or surgical center prefers that the tools be organized and prepared prior to surgery. In FIG. 17A, the surgical setup images include the illustration of a Mayo Stand that depicts a collection of tools laid out on the Mayo Stand.

Turning now to FIG. 17B, the user interface 1700 includes further listings of supplies 1708 needed for the surgery. The user interface 1700 includes additional surgical setup images 1710, and in the example illustrated in FIG. 17B, the surgical setup images 1710 include a depiction of a back table with an assortment of tools and supplies set up on the back table. The user interface 1700 includes a preparation and positioning 1712 section for providing information about how the patient should be prepared for surgery. The user interface 1700 includes a medications and dressing 1714 section for providing information about how the patient should be prepared for surgery and dressed after surgery.

Turning now to FIG. 17C, the user interface 1700 includes further listings of supplies 1708 needed for the surgery. The user interface 1700 includes further surgical setup images 1710 depicting how the operation room should be prepared for surgery. The surgical setup image 1710 illustrated in FIG. 17C depicts a complete room setup with a depiction of several tables comprising surgical tools and supplies. The user interface 1700 includes a requested music 1716 section providing the surgeon's requested music during the surgery.

Turning now to FIGS. 17D-17E, the user interface 1700 includes further listings of supplies 1708 needed for the surgery. The supplies 1708 may include, for example, supplies and custom packs, instruments, trays, equipment, gloves, sutures, implantable items, medications, and so forth. The listing of supplies 1708 may include an image of the requested item, a surgical center-specific price for the item, a predicted price for the item, a reference number for the item, and indication of how the item is priced (i.e., per-unit, per-bottle, and so forth), a quantity for how many of the requested item should be present in the surgery, an indication of how many of the requested item have been held or reserved for the surgery, and so forth.

The user interface 1700 is accessible by way of a web browser or application. The user interface 1700 enables a user to hover over a certain item and receive additional information about that item when the cursor is hovering over the item. In an example implementation, when a user hovers over a certain item, a popup may appear indicating whether the item is expensive. In an instance where the item is priced higher than usual, a popup may appear indicating that the specific item is expensive, and the user should consider holding that item rather than immediately opening the item. In some cases, it may be desirable to leave some items unopened until they are necessary during the surgery. In some cases, the item might not be opened, and then the patient and healthcare provider can save on the cost of the item.

The user interface 1700 includes hyperlinks enabling a user to gather additional information about a certain category. The surgical setup images 1710 include a hyperlink for viewing a high-resolution version of the image. The listing of supplies 1708 include hyperlinks for expanding the item to view more information about the item; viewing a database output indicating how many of the certain item is in-stock at the surgical center; purchasing the requested item directly from a supplier; or requesting the item from a pharmacy or other office at the surgical center. The user interface 1700 includes hyperlinks for accessing additional information about, for example, the names of the items, the cost of the items, the reference numbers for the items, the unit-types for the items, whether a certain quantity of the items are open, and whether a certain quantity of the items are held.

The user interface 1700 includes a means to directly contact any of the surgeon, surgical staff, surgical center, healthcare provider, and so forth. The contact information may include a means to initiate a telephone call, video chat, or Internet-based phone call. The contact information may include a means to email, text message, or otherwise communicate with any listed person. The contact information may include information about the patient.

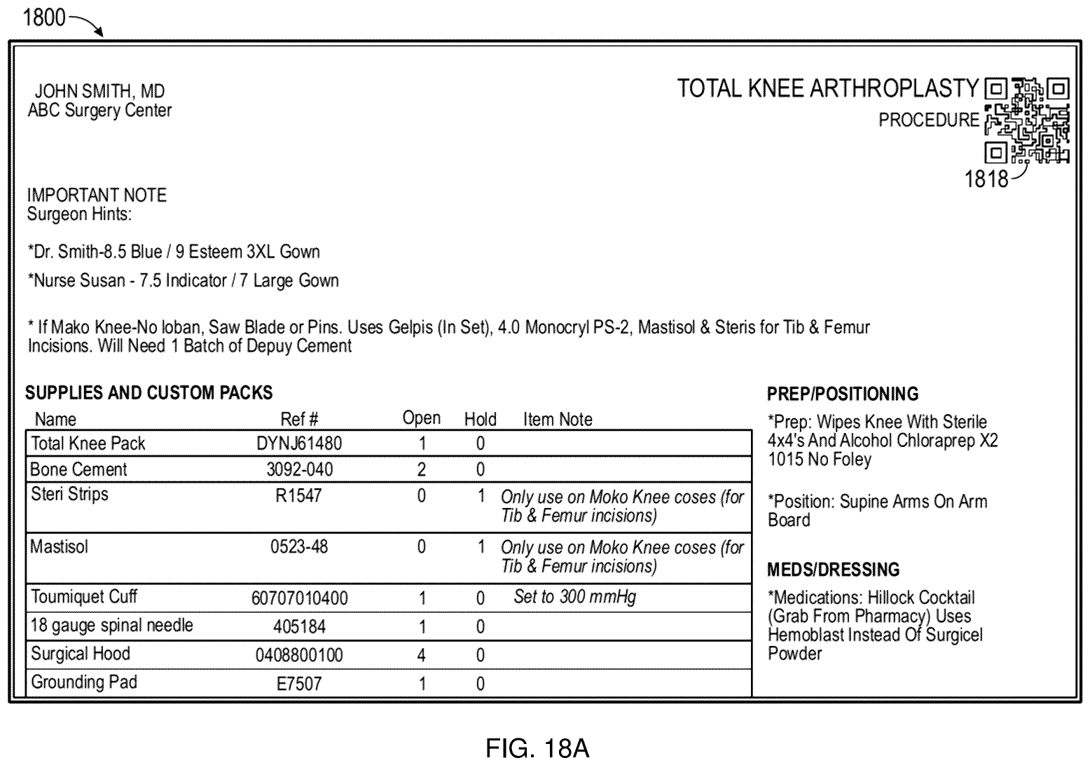

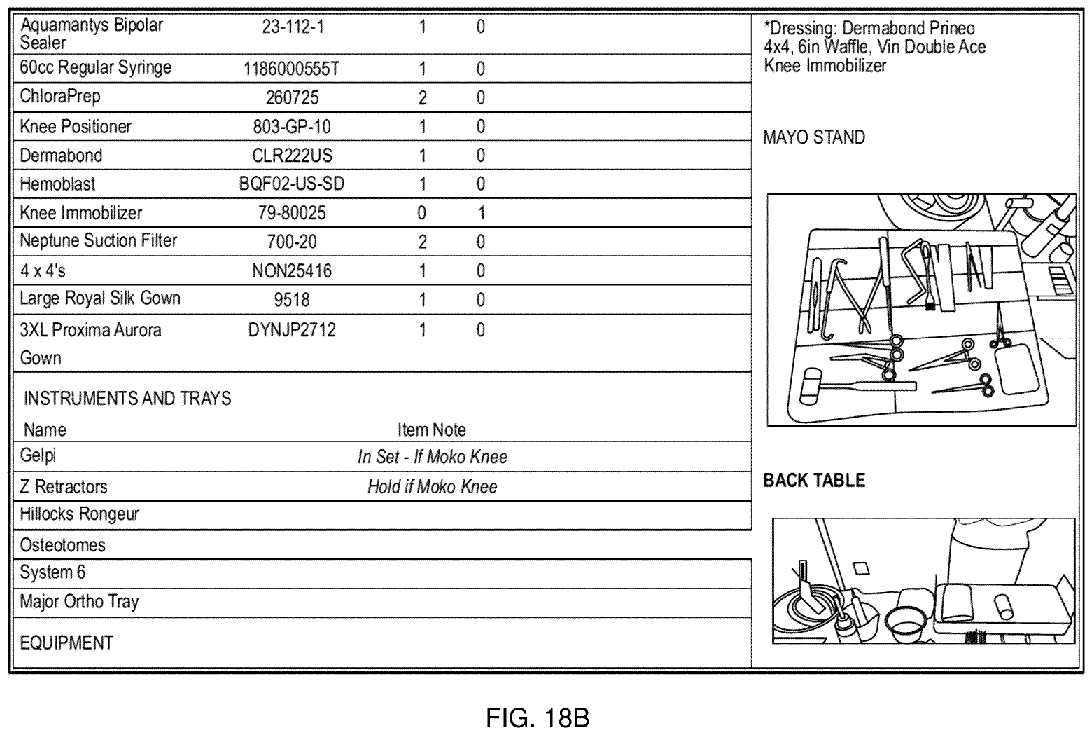

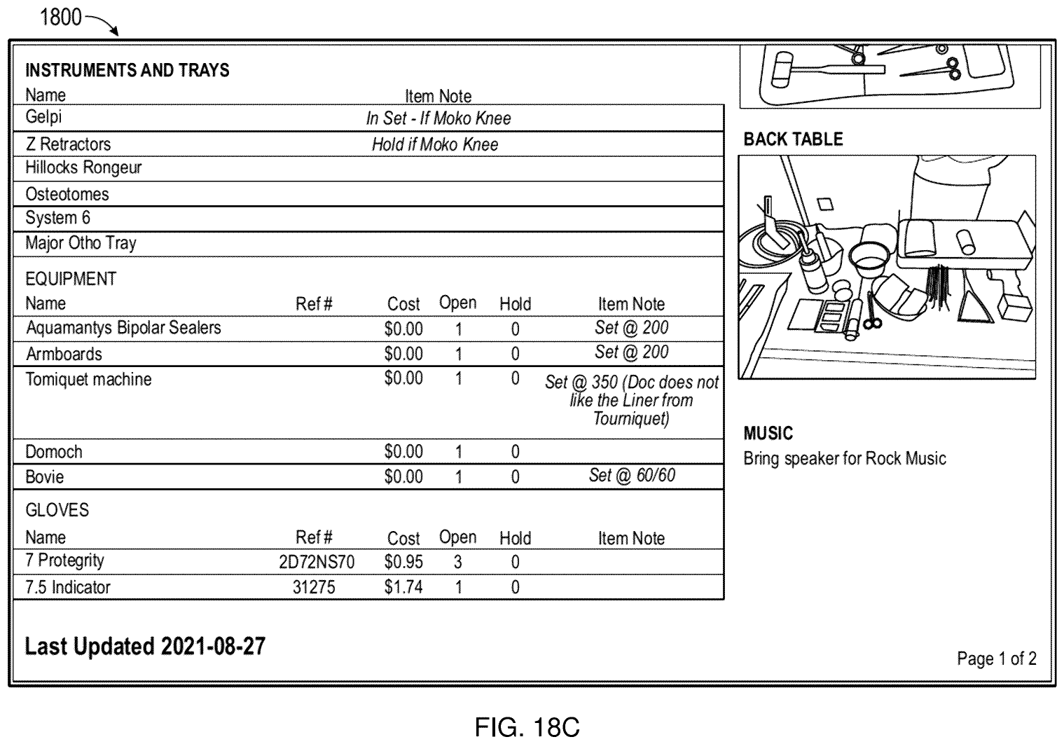

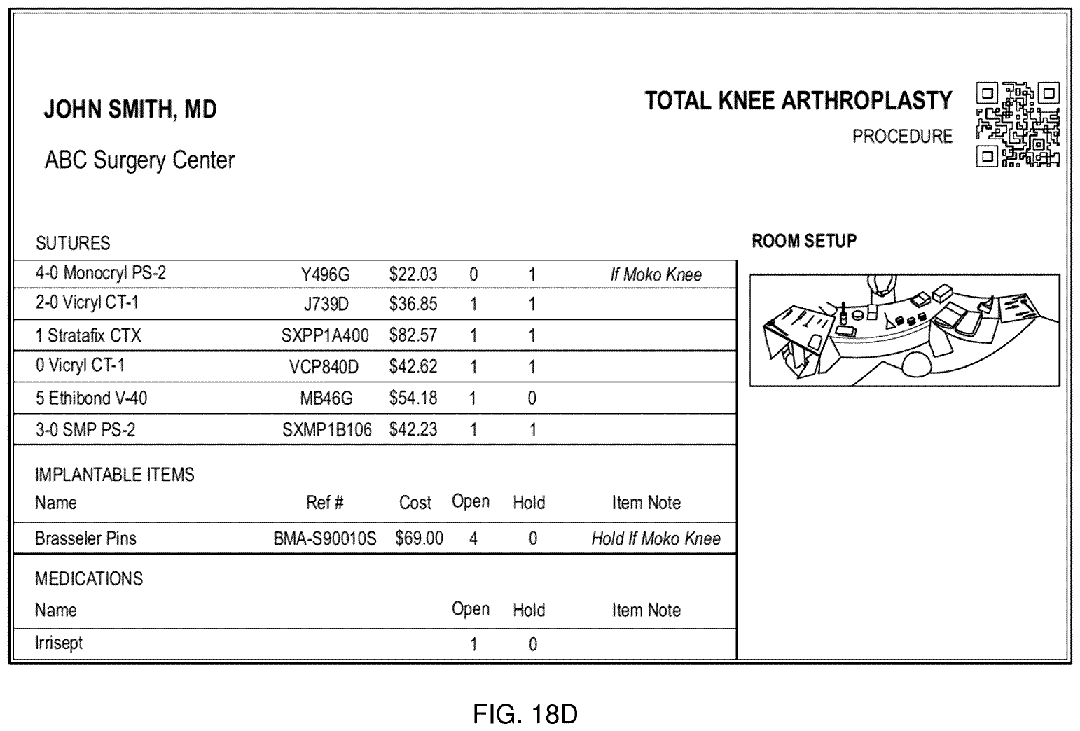

FIGS. 18A-18D illustrate screenshots of an example virtual file 1800 for presenting an aggregated data form. The virtual file 1800 may be downloaded from the user interface 1700. The example virtual file illustrated in FIGS. 18A-18D includes the same information viewable in the user interface 1700 illustrated in FIGS. 17A-17E. The virtual file 1800 includes a unique code 1818. The unique code 1818 may include a QR code as illustrated in FIGS. 18A-18D or may include some other unique code.

The unique code 1818 may be any scannable figure or code that is readable by the personal device 108 such as a mobile phone, camera, or other device comprising an image sensor. In an embodiment, the unique code 1818 is a two-dimensional barcode such as a quick response (QR) code. The two-dimensional barcode can be digitally scanned by a camera or other sensor on the personal device 108. The unique code 1818 may include multiple squares that can be read by the image sensor of the personal device 108.

In an embodiment where the unique code 1818 is a QR code, the code includes three large squares (the three large squares can be seen in the upper-left, lower-left, and upper-right corners of the example unique code 1818 shown in FIGS. 18A-18D) that serve as alignment targets while a smaller square in a remaining corner of the unique code 1818 (the smaller square can be seen near the lower-right corner of the example unique code 1818 shown in FIGS. 18A-18D) serves to normalize the angle with which the image sensor hits the unique code 1818. The remaining area of the unique code 1818 is the actual data that is converted into binary code by the personal device 108. The unique code 1818 may include many characters worth of data. In an example where the unique code 1818 is a 117-pixel square, the code may hold 1852 characters of data.

In an embodiment, an image sensor of the personal device 108 is directed to scan the unique code 1818, and the unique code 1818 includes instructions for the personal device 108 to connect to the data aggregation server 104. A processor of the personal device 108 may execute the instructions stored in the unique code 1818 to automatically connect to the data aggregation server 104. In various implementations, the personal device 108 may request permission from a user and/or query the user whether the personal device 108 should connect to the media server 104. In an embodiment, automatically connecting to the data aggregation server 104 brings the data aggregation platform 102 up on the personal device 108 in an application, program, webpage, or by some other suitable means.

When a user connects to the data aggregation platform 102 upon scanning the unique code 1818, the user may be directed to the user interface 1700 such as the one illustrated in FIGS. 17A-17E. The user may then view details about the aggregated data form, propose amendments to the aggregated data form, correct errors in the aggregated data form, initiate contact with one or more persons associated with the aggregated data form, and so forth. The user may additionally view other aggregated data forms saved on the data aggregation server 104 associated with the same surgeon, surgical center, healthcare network, healthcare facility, and so forth. The user may view public aggregated data forms posted to the data aggregation server 104. The user may select a case, build out the card, add or remove items to the card, document usage of the card, enter surgical notes, and so forth.

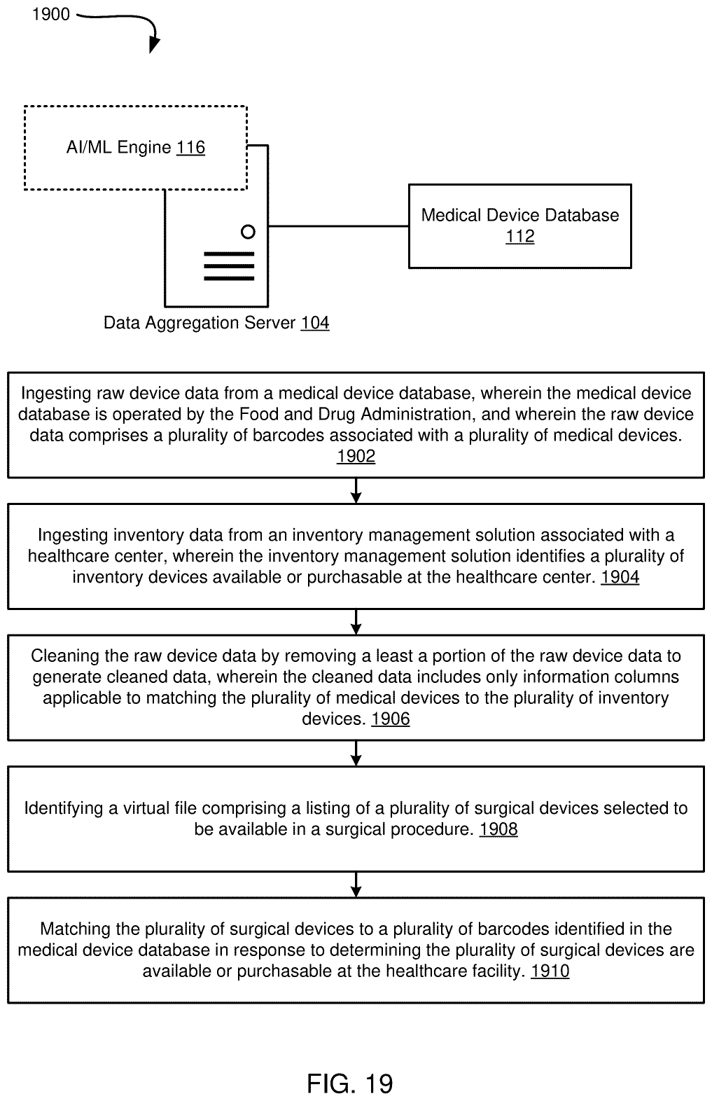

FIG. 19 is a schematic flow chart diagram of a method 1900 matching barcodes to medical devices that included on an aggregated data form. The method 1900 is performed by the data aggregation server 104.

The method 1900 includes ingesting at 1902 raw device data from a medical device database, wherein the medical device database is operated by the Food and Drug Administration, and wherein the raw device data comprises a plurality of barcodes associated with a plurality of medical devices. The method 1900 includes ingesting at 1904 inventory data from an inventory management solution associated with a healthcare center, wherein the inventory management solution identifies a plurality of inventory devices available or purchasable at the healthcare center. The method 1900 includes cleaning at 1906 the raw device data by removing at least a portion of the raw device data to generate cleaned data, wherein the cleaned data includes only information columns applicable to matching the plurality of medical devices to the plurality of inventory devices. The method 1900 includes identifying at 1908 a virtual file comprising a listing of a plurality of surgical devices selected to be available in a surgical procedure. The method 1900 includes matching at 1910 the plurality of surgical devices to a plurality of barcodes identified in the medical device database in response to determining the plurality of surgical devices are available or purchasable at the healthcare facility.

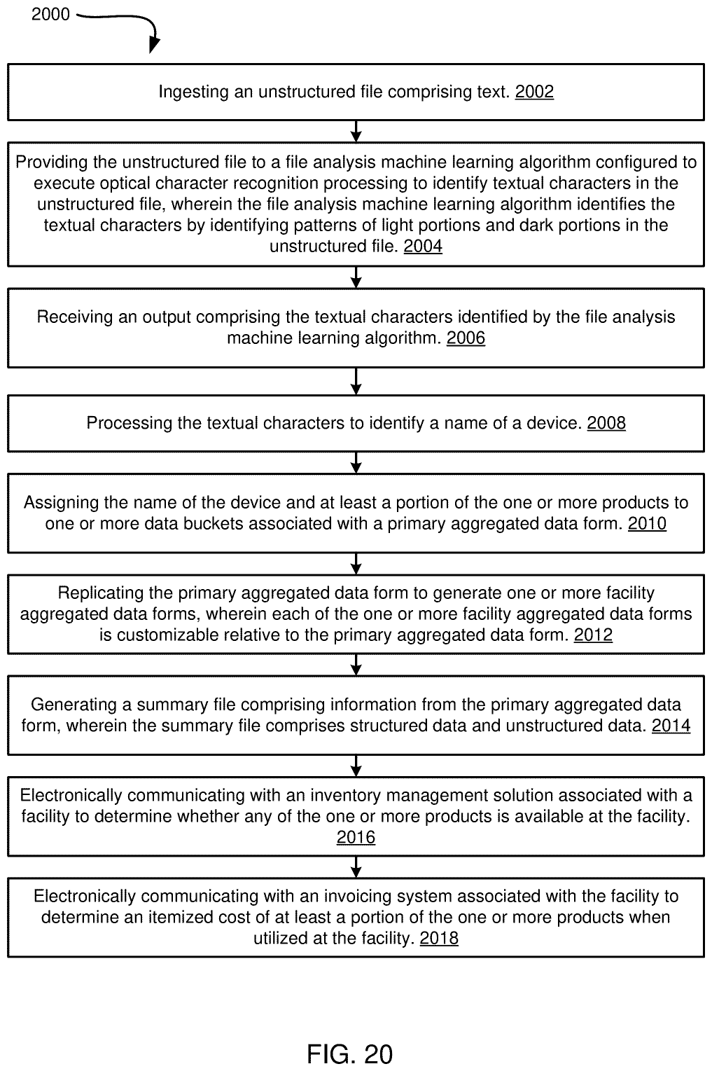

FIG. 20 is a schematic flow chart diagram of a method 2000 for generating an aggregated data form. The method 2000 is performed by the data aggregation server 104. The method 2000 includes ingesting at 2002 an unstructured file comprising text. The method 2000 includes providing at 2004 the unstructured file to a file analysis machine learning algorithm configured to execute optical character recognition processing to identify textual characters in the unstructured file, wherein the file analysis machine learning algorithm identifies the textual characters by identifying patterns of light portions and dark portions in the unstructured file. The method 2000 includes receiving at 2006 an output comprising the textual characters identified by the file analysis machine learning algorithm. The method 2000 includes processing at 2008 the textual characters to identify a name of a device. The method 2000 includes assigning at 2010 the name of the device and at least a portion of the one or more products to one or more data buckets associated with a primary aggregated data form. The method 2000 includes replicating at 2012 the primary aggregated data form to generate one or more facility aggregated data forms, wherein each of the one or more facility aggregated data forms is customizable relative to the primary aggregated data form. The method 2000 includes generating at 2014 a summary file comprising information from the primary aggregated data form, wherein the summary file comprises structured data and unstructured data. The method 2000 includes electronically communicating at 2016 with an inventory management solution associated with a facility to determine whether any of the one or more products is available at the facility. The method 2000 includes electronically communicating at 2018 with an invoicing system associated with the facility to determine an itemized cost of at least a portion of the one or more products when utilized at the facility.

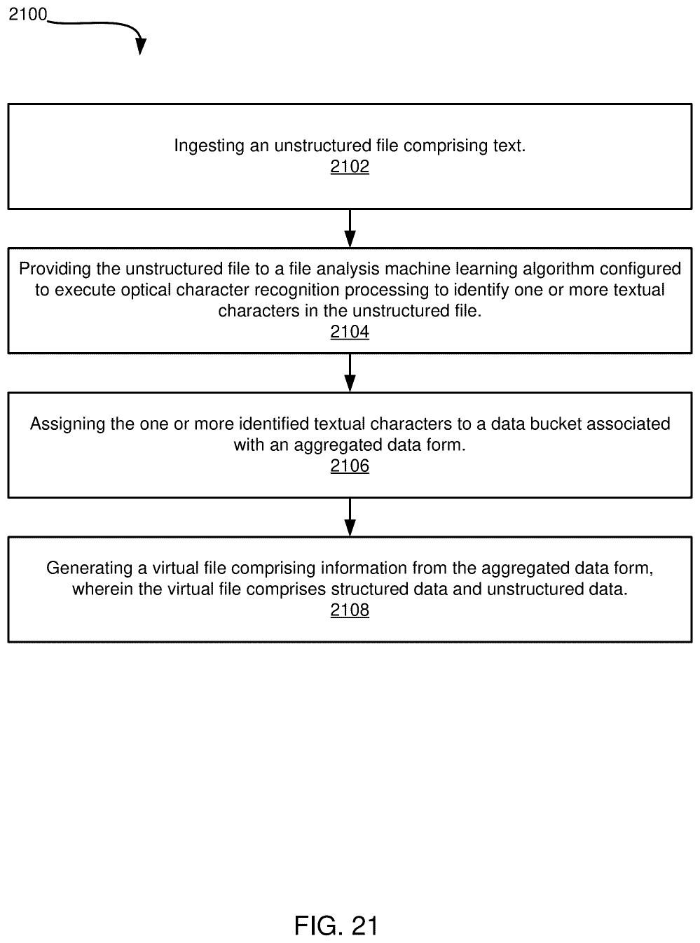

FIG. 21 is a schematic flow chart diagram of a method 2100 for performing file analysis and classifying information in data buckets associated with an aggregated data form. The method 2100 may be performed by a computing device, such as the data aggregation server 104 and/or computing components associated with the data aggregation platform 102 as described herein.

The method 2100 begins and a data ingestion engine ingests at 2102 an unstructured file comprising text. The unstructured file may include, for example, an image, video file, scan, PDF, Microsoft® Word document, and so forth. The method 2100 continues and a computing processor provides at 2104 the unstructured file to a file analysis machine learning algorithm configured to execute optical character recognition processing to identify one or more textual characters in the unstructured file. The method 2100 continues and a computing processor assigns at 2106 the one or more identified textual characters to a data bucket associated with an aggregated data form. The method 2100 continues and a computing processor generates at 2108 a virtual file comprising information from the aggregated data form, wherein the virtual file comprises structured data and unstructured data.

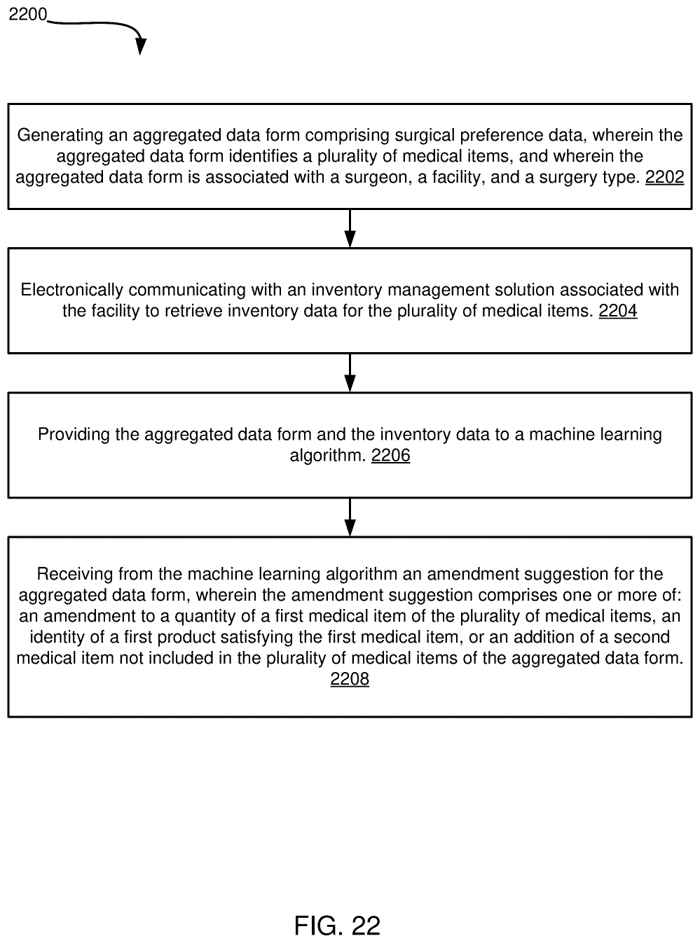

FIG. 22 is a schematic flow chart diagram of a method 2200 for leveraging an AI/ML engine 116 of a data aggregation server 104 to provide amendment suggestions for an aggregated data form. The method 2200 includes generating at 2202 an aggregated data form comprising surgical preference data, wherein the aggregated data form identifies a plurality of medical items, and wherein the aggregated data form is associated with a surgeon, a facility, and a surgery type. The method 2200 includes electronically communicating at 2204 with an inventory management solution associated with the facility to retrieve inventory data for the plurality of medical items. The method 2200 includes providing at 2206 the aggregated data form and the inventory data to a machine learning algorithm. The method 2200 includes receiving at 2208 from the machine learning algorithm an amendment suggestion for the aggregated data form, wherein the amendment suggestion comprises one or more of: an amendment to a quantity of a first medical item; an identity of a first product satisfying the first medical item; or an addition of a second medical item not included in the aggregated data form. The machine learning algorithm is trained on the inventory data and further on a plurality of aggregated data forms associated with one or more of the same surgeon, the same facility, or the same surgery type.

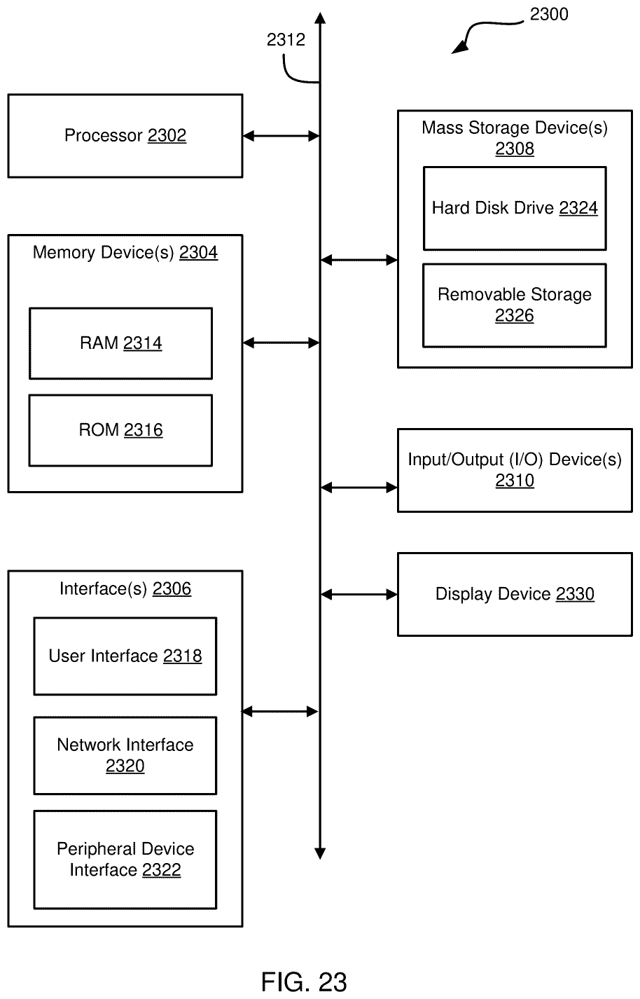

Referring now to FIG. 23, a block diagram of an example computing device 2300 is illustrated. Computing device 2300 may be used to perform various procedures, such as those discussed herein. Computing device 2300 can perform various monitoring functions as discussed herein, and can execute one or more application programs, such as the application programs or functionality described herein. Computing device 2300 can be any of a wide variety of computing devices, such as a desktop computer, in-dash computer, vehicle control system, a notebook computer, a server computer, a handheld computer, tablet computer and the like.

Computing device 2300 includes one or more processor(s) 2304, one or more memory device(s) 2304, one or more interface(s) 2306, one or more mass storage device(s) 2308, one or more Input/Output (I/O) device(s) 2310, and a display device 2330 all of which are coupled to a bus 2312. Processor(s) 2304 include one or more processors or controllers that execute instructions stored in memory device(s) 2304 and/or mass storage device(s) 2308. Processor(s) 2304 may also include various types of computer-readable media, such as cache memory.

Memory device(s) 2304 include various computer-readable media, such as volatile memory (e.g., random access memory (RAM) 2314) and/or nonvolatile memory (e.g., read-only memory (ROM) 2316). Memory device(s) 2304 may also include rewritable ROM, such as Flash memory.

Mass storage device(s) 2308 include various computer readable media, such as magnetic tapes, magnetic disks, optical disks, solid-state memory (e.g., Flash memory), and so forth. As shown in FIG. 23, a particular mass storage device 2308 is a hard disk drive 2324. Various drives may also be included in mass storage device(s) 2308 to enable reading from and/or writing to the various computer readable media. Mass storage device(s) 2308 include removable media 2326 and/or non-removable media.

I/O device(s) 2310 include various devices that allow data and/or other information to be input to or retrieved from computing device 2300. Example I/O device(s) 2310 include cursor control devices, keyboards, keypads, microphones, monitors or other display devices, speakers, printers, network interface cards, modems, and the like.

Display device 2330 includes any type of device capable of displaying information to one or more users of computing device 2300. Examples of display device 2330 include a monitor, display terminal, video projection device, and the like.

Interface(s) 2306 include various interfaces that allow computing device 2300 to interact with other systems, devices, or computing environments. Example interface(s) 2306 may include any number of different network interfaces 2320, such as interfaces to local area networks (LANs), wide area networks (WANs), wireless networks, and the Internet. Other interface(s) include user interface 2318 and peripheral device interface 2322. The interface(s) 2306 may also include one or more user interface elements 2318. The interface(s) 2306 may also include one or more peripheral interfaces such as interfaces for printers, pointing devices (mice, track pad, or any suitable user interface now known to those of ordinary skill in the field, or later discovered), keyboards, and the like.

Bus 2312 allows processor(s) 2304, memory device(s) 2304, interface(s) 2306, mass storage device(s) 2308, and I/O device(s) 2310 to communicate with one another, as well as other devices or components coupled to bus 2312. Bus 2312 represents one or more of several types of bus structures, such as a system bus, PCI bus, IEEE bus, USB bus, and so forth.

For purposes of illustration, programs and other executable program components are shown herein as discrete blocks, such as block 302 for example, although it is understood that such programs and components may reside at various times in different storage components of computing device 2300 and are executed by processor(s) 2302. Alternatively, the systems and procedures described herein, including programs or other executable program components, can be implemented in hardware, or a combination of hardware, software, and/or firmware. For example, one or more application specific integrated circuits (ASICs) can be programmed to carry out one or more of the systems and procedures described herein.

### EXAMPLES

The following examples pertain to further implementations of the disclosure.

Example 1 is a method. The method includes receiving an indication of a surgical procedure to be performed. The method includes identifying one or more suggested items to be used during the surgical procedure. The method includes generating an aggregated data form for the surgical procedure, wherein the aggregated data form comprises a listing of the one or more suggested items.

Example 2 is a method as in Example 1, further comprising generating a unique code associated with the surgical procedure, wherein the unique code is scannable and comprises instructions for connecting to a server supporting the aggregated data form.

Example 3 is a method as in any of Examples 1-2, wherein identifying the one or more suggested items comprises providing the surgical procedure to an AI/ML engine configured to predict the one or more suggested items based on a plurality of surgical procedures performed by a plurality of healthcare providers.

Example 4 is a method as in any of Examples 1-3, wherein the aggregated data form comprising real-time pricing for each of the one or more suggested items.

Example 5 is a method as in any of Examples 1-4, further comprising communicating the aggregated data form to an automated billing system for identifying one or more items used during the surgical procedure.

Example 6 is a method as in any of Examples 1-5, further comprising associating the aggregated data form with a healthcare provider and providing read and write access on the aggregated data form to the healthcare provider.

Example 7 is a method as in any of Examples 1-6, further comprising associating one or more surgical setup images with the aggregated data form, wherein the surgical setup images comprise a depiction of how items should be arranged prior to the surgical procedure.

Example 8 is a method as in any of Examples 1-7, further comprising determining a minimum quantity and a maximum quantity for each of the one or more suggested items.

Example 9 is a method of collecting, aggregating, and analyzing data, the method comprising. The method includes ingesting an unstructured file comprising text. The method includes providing the unstructured file to a file analysis machine learning algorithm configured to execute optical character recognition processing to identify one or more textual characters in the unstructured file. The method includes assigning the one or more identified textual characters to a data bucket associated with an aggregated data form. The method includes generating a virtual file comprising information from the aggregated data form, wherein the virtual file comprises structured data and unstructured data.

Example 10 is a method as in Example 9, wherein: the aggregated data form comprises a plurality of independent data buckets; each of the plurality of independent data buckets is configured to receive data of a certain file type and a certain content; and the process of assigning the one or more identified textual characters to the data bucket associated with the aggregated data form comprises identifying a correct data bucket for the one or more identified textual characters.

Example 11 is a method as in any of Examples 9-10, wherein generating the virtual file comprises generating a summary file comprising the structured data and the unstructured data stored in the aggregated data form, wherein the summary file comprises one or more of a collection of data accessible on a user interface, a collection of data accessible on a web page, or a file stored in a portable document format.

Example 12 is a method as in any of Examples 9-11, further comprising communicating with a web scraping module, wherein the web scraping module is configured to extract structured data from one or more web pages, wherein the structured data comprises information regarding one or more products, the information comprising one or more of a name, unique product code, regulatory approval status, current price, current availability, or safety rating.

Example 13 is a method as in any of Examples 9-12, wherein the aggregated data form comprises information for a surgical preference card, and wherein the method further comprises: storing a master aggregated data form associated with a healthcare system, wherein the healthcare system comprises a plurality of surgical facilities; storing a plurality of facility aggregated data forms, wherein each of the plurality of facility aggregated data forms is associated with one facility of the plurality of surgical facilities in the healthcare system; updating each of the plurality of facility aggregated data forms when the master aggregated data form is updated.

Example 14 is a method as in any of Examples 9-13, wherein the optical character recognition processing performed by the file analysis machine learning algorithm comprises: identifying a boundary between a bright pixel region and a dark pixel region; classifying the bright pixel region as background; processing the dark pixel region to identify a textual character, wherein the textual character comprises one or more of a letter, number, emoji, or punctuation character.

Example 15 is a method as in any of Examples 9-14, wherein the aggregated data form comprises a plurality of data buckets, and wherein one or more of the plurality of data buckets is a required data bucket that must be filled with data for the aggregated data form to be complete, and wherein the method further comprises: determining whether the aggregated data form is complete based on whether each of the required data buckets is filled; and in response to determining that each of the required data buckets is filled, generating a message querying a user whether the aggregated data bucket is complete.

Example 16 is a method as in any of Examples 9-15, wherein the aggregated data form comprises digital information for a surgical preference card, and wherein the unstructured file comprises a depiction of a completed preference card, and wherein the depiction of the completed preference card comprises one or more of: an image, scan, or portable document format (PDF) of a handwritten surgical preference card; an image, scan, or PDF of a printed surgical preference card; or an image, scan, or PDF of a digital surgical preference card.

Example 17 is a method as in any of Examples 9-16, further comprising rendering a user interface accessible to a user, wherein the user interface comprises: a dropdown listing of a plurality of items listed on a surgical preference card, wherein the dropdown listing comprises functionality enabling the user to expand or diminish the dropdown listing; a hyperlink associated with one or more of the plurality of items in the dropdown listing, wherein the hyperlink directs to a means for ordering the corresponding item; a text box associated with one or more of the plurality of items in the dropdown listing, wherein the text box comprises a means for a user to input a quantity of the corresponding item; and a button associated with one or more of the plurality of items in the dropdown listing, wherein the button comprises a means for a user to delete the corresponding item from the surgical preference card.

Example 18 is a method as in any of Examples 9-17, further comprising: determining whether the aggregated data form is a master aggregated data form, or a facility aggregated data form; in response to determining the aggregated data form is a master aggregated data form, identifying one or more facility aggregated data forms that are associated with the master aggregated data form; determining whether any of the one or more facility aggregated data forms is inconsistent with information stored on the master aggregated data form; and in response to determining that at least one of the one or more facility aggregated data forms is inconsistent with the master aggregated data form, updating the at least one facility aggregated data form to comprise the same data associated with the master aggregated data form.

Example 19 is a system comprising one or more processors for executing instructions stored in non-transitory computer readable storage medium, wherein the instructions comprising any of the method steps described in connection with Examples 1-19.

Example 20 is non-transitory computer readable storage medium storing instructions to be executed by one or more processors, the instructions comprising any of the method steps described in connection with Examples 1-19.

Example 21 is means for performing any of the method steps described in connection with Examples 1-19.

Example 22 is a system. The system includes means for ingesting an unstructured file comprising text. The system includes means for providing the unstructured file to a file analysis machine learning algorithm configured to execute optical character recognition processing to identify one or more textual characters in the unstructured file. The system includes means for assigning the one or more identified textual characters to a data bucket associated with an aggregated data form. The system includes means for generating a virtual file comprising information from the aggregated data form, wherein the virtual file comprises structured data and unstructured data.

Example 23 is a system as in Example 22, wherein: the aggregated data form comprises a plurality of independent data buckets; each of the plurality of independent data buckets is configured to receive data of a certain file type and a certain content; and the means for assigning the one or more identified textual characters to the data bucket associated with the aggregated data form comprises means for identifying a correct data bucket for the one or more identified textual characters.

Example 24 is a system as in any of Examples 22-23, wherein the means for generating the virtual file comprises means for generating a summary file comprising the structured data and the unstructured data stored in the aggregated data form, wherein the summary file comprises one or more of a collection of data accessible on a user interface, a collection of data accessible on a web page, or a file stored in a portable document format.

Example 25 is a system as in any of Examples 22-24, further comprising means for communicating with a web scraping module, wherein the web scraping module is configured to extract structured data from one or more web pages, wherein the structured data comprises information regarding one or more products, the information comprising one or more of a name, unique product code, regulatory approval status, current price, current availability, or safety rating.

Example 26 is a system as in any of Examples 22-25, wherein the aggregated data form comprises information for a surgical preference card, and wherein the system further comprises: means for storing a master aggregated data form associated with a healthcare system, wherein the healthcare system comprises a plurality of surgical facilities; means for storing a plurality of facility aggregated data forms, wherein each of the plurality of facility aggregated data forms is associated with one facility of the plurality of surgical facilities in the healthcare system; and means updating each of the plurality of facility aggregated data forms when the master aggregated data form is updated.

Example 27 is a system as in any of Examples 22-26, wherein the file analysis machine learning algorithm comprises: means for identifying a boundary between a bright pixel region and a dark pixel region; means for classifying the bright pixel region as background; and means for processing the dark pixel region to identify a textual character, wherein the textual character comprises one or more of a letter, number, emoji, or punctuation character.

Example 28 is a system as in any of Examples 22-27, wherein the aggregated data form comprises a plurality of data buckets, and wherein one or more of the plurality of data buckets is a required data bucket that must be filled with data for the aggregated data form to be complete, and wherein the system further comprises: means for determining whether the aggregated data form is complete based on whether each of the required data buckets is filled; and means for generating a message querying a user whether the aggregated data bucket is complete in response to determining that each of the required data buckets is filled.

Example 29 is a system as in any of Examples 22-28, wherein the aggregated data form comprises digital information for a surgical preference card, and wherein the unstructured file comprises a depiction of a completed preference card, and wherein the depiction of the completed preference card comprises one or more of: an image, scan, or portable document format (PDF) of a handwritten surgical preference card; an image, scan, or PDF of a printed surgical preference card; or an image, scan, or PDF of a digital surgical preference card.

Example 30 is a system as in any of Examples 22-29, further comprising means for rendering a user interface accessible to a user, wherein the user interface comprises: a dropdown listing of a plurality of items listed on a surgical preference card, wherein the dropdown listing comprises functionality enabling the user to expand or diminish the dropdown listing; a hyperlink associated with one or more of the plurality of items in the dropdown listing, wherein the hyperlink directs to a means for ordering the corresponding item; a text box associated with one or more of the plurality of items in the dropdown listing, wherein the text box comprises a means for a user to input a quantity of the corresponding item; and a button associated with one or more of the plurality of items in the dropdown listing, wherein the button comprises a means for a user to delete the corresponding item from the surgical preference card.

Example 31 is a system as in any of Examples 22-30, further comprising: means for determining whether the aggregated data form is a master aggregated data form, or a facility aggregated data form; means for identifying one or more facility aggregated data forms that are associated with the master aggregated data form in response to determining the aggregated data form is a master aggregated data form; means for determining whether any of the one or more facility aggregated data forms is inconsistent with information stored on the master aggregated data form; and means for updating the at least one facility aggregated data form to comprise the same data associated with the master aggregated data form in response to determining that at least one of the one or more facility aggregated data forms is inconsistent with the master aggregated data form.

Example 32 is a method as in any of Examples 1-17. The method includes generating an aggregated data form comprising surgical preference data, wherein the aggregated data form identifies a plurality of medical items, and wherein the aggregated data form is associated with a surgeon, a facility, and a surgery type. The method includes electronically communicating with an inventory management solution associated with the facility to retrieve inventory data for the plurality of medical items. The method includes providing the aggregated data form and the inventory data to a machine learning algorithm. The method includes receiving from the machine learning algorithm an amendment suggestion for the aggregated data form, wherein the amendment suggestion comprises one or more of: an amendment to a quantity of a first medical item; an identity of a first product satisfying the first medical item; or an addition of a second medical item not included in the aggregated data form. The method is such that the machine learning algorithm is trained on the inventory data and further on a plurality of aggregated data forms associated with one or more of the same surgeon, the same facility, or the same surgery type.

Example 33 is a method as in Example 32 or any of Examples 1-17, further comprising: ingesting an unstructured file comprising text; providing the unstructured file to a file analysis machine learning algorithm configured to execute optical character recognition processing to identify textual characters in the unstructured file, wherein the file analysis machine learning algorithm identifies the textual characters by identifying patterns of light portions and dark portions in the unstructured file; receiving an output comprising the textual characters identified by the file analysis machine learning algorithm; processing the textual characters to identify a plurality of names for the plurality of medical items; assigning the plurality of medical items to the aggregated data form, and storing the aggregated data form on a cloud-based database; and generating a summary file comprising information from the aggregated data form, wherein the summary file comprises structured data and unstructured data.

Example 34 is a method as in any of Examples 1-17 or Examples 32-33, wherein the inventory data comprises surgical usage data for the facility, and wherein the surgical usage data comprises an indication of which products were utilized in a plurality of surgical procedures performed at the facility.

Example 35 is a method as in any of Examples 1-17 or Examples 32-34, wherein the machine learning algorithm is trained to assess the surgical usage data for the facility; wherein the amendment suggestion output by the machine learning algorithm comprises the amendment to the quantity of the first medical item; and wherein the machine learning algorithm suggests the amendment to the quantity based upon one or more of: determining the same surgeon historically utilizes more of the first medical item or fewer of the first medical item when performing the surgery type at the facility, as determined based upon the surgical usage data; determining that any surgeon at the facility historically utilizes more of the first medical item or fewer of the first medical item when performing the surgery type at the facility, as determined based upon the surgical usage data; or determining that any of a plurality of surgeons request more of the first medical item or fewer of the first medical item when performing the surgery type, as determined based upon a plurality of aggregated data forms associated with the plurality of surgeons.

Example 36 is a method as in any of Examples 1-17 or Examples 32-35, wherein the machine learning algorithm suggests the first product satisfying the first medical item based upon one or more of: determining the first product is available at the facility, as determined based upon the inventory data; determining the first product is a most affordable product satisfying the first medical item available at the facility, as determined based upon the inventory data; or determining the first product satisfies the first medical item, as determined based upon medical device data ingested from a medical device database.

Example 37 is a method as in any of Examples 1-17 or Examples 32-36, wherein the machine learning algorithm suggests the first product satisfying the first medical item based upon determining the first product is utilized to satisfy the first medical item by one or more other surgeons performing the same surgery type, as determined based upon a plurality of aggregated data forms associated with the plurality of surgeons.

Example 38 is a method as in any of Examples 1-17 or Examples 32-37, wherein the machine learning algorithm suggests the addition of the second medical item based upon determining the second medical item is utilized to perform the same surgery type by one or more of a plurality of other surgeons, as determined based upon a plurality of aggregated data forms associated with the plurality of other surgeons.

Example 39 is a method as in any of Examples 1-17 or Examples 32-38, further comprising: ingesting medical device data from a medical device database, wherein the medical device data comprises a listing of a plurality of medical devices approved for use at the facility; ingesting pharmaceutical data from a pharmaceutical database, wherein the pharmaceutical data comprises a listing of a plurality of pharmaceuticals approved for use at the facility; matching each of the plurality of medical items identified on the aggregated data from with at least one medical device of the plurality of medical devices, or at least one pharmaceutical of the plurality of pharmaceuticals.

Example 40 is a method as in any of Examples 1-17 or Examples 32-39, further comprising: storing data for a plurality of medical devices on a database, wherein the data includes a barcode associated with each of the plurality of medical devices; determining that a first medical device of the plurality of medical devices satisfies the first medical item identified in the aggregated data form; receiving an indication that a barcode scanner scanned a first barcode associated with the first medical device in preparation for performing the surgery type; matching the first barcode with the first medical device; matching the first medical device with the aggregated data form; and marking the first medical item on the aggregated data form as having been collected in preparation for performing the surgery type.

Example 41 is a method as in any of Examples 1-17 or Examples 32-40, further comprising generating a scannable code associated with the surgical preference data, wherein the scannable code redirects to one or more of: a complete version of the aggregated data form comprising all information associated with the aggregated data form; or a cleaned version of the aggregated data form that does not comprise personal health information.

Example 42 is a method as in any of Examples 1-17 or Examples 32-41, further comprising: storing a plurality of aggregated data forms on a database, wherein each of the plurality of aggregated data forms is associated with the facility; and exporting each of the plurality of aggregated data forms to a compressed file to be stored on local memory at the facility; wherein the compressed file is accessible at the facility in the event of a network outage.

Example 43 is a method as in any of Examples 1-17 or Examples 32-42, further comprising communicating with a web scraping module, wherein the web scraping module is configured to extract structured data from one or more web pages, wherein the structured data comprises information regarding one or more medical products, the information comprising one or more of a name, unique product code, regulatory approval status, current price, current availability, or safety rating.

Example 44 is a method as in any of Examples 1-17 or Examples 32-43, wherein the aggregated data form comprises a plurality of data buckets, and wherein one or more of the plurality of data buckets is a required data bucket that must be filled with data for the aggregated data form to be complete, and wherein the method further comprises: determining whether the aggregated data form is complete based on whether each of the required data buckets is filled; and in response to determining that each of the required data buckets is filled, generating a message querying a user whether the aggregated data bucket is complete.

Example 45 is a method as in any of Examples 1-17 or Examples 32-44, further comprising: storing a plurality of aggregated data forms on a cloud-based database, wherein the plurality of aggregated data forms is associated with the same facility and the same surgery type; and rendering a user interface accessible to a user when the user is preparing or viewing the aggregated data form, wherein the user interface comprises, for each of the plurality of medical items on the aggregated data form, an indication of a proportion of the plurality of aggregated data forms that includes the corresponding medical item.

Example 46 is a method as in any of Examples 1-17 or Examples 32-45, further comprising: storing a plurality of aggregated data forms on a cloud-based database, wherein the plurality of aggregated data forms is associated with the same facility and the same surgery type; and rendering a user interface accessible to a user when the user is preparing or viewing the aggregated data form, wherein the user interface comprises a suggestion of additional medical items to be included in the aggregated data form; wherein the additional medical items are included in at least a portion of the plurality of aggregated data form.

Example 47 is a method as in any of Examples 1-17 or Examples 32-46, wherein at least one of the plurality of medical items is a special handling item, and wherein the special handling item is not included in the inventory management solution associated with the facility.

Example 48 is a method as in any of Examples 1-17 or Examples 32-47, wherein the plurality of medical items comprises one or more of medical devices or pharmaceuticals to be present when the surgeon performs the surgery type at the facility.

Example 49 is a method as in any of Examples 1-17 or Examples 32-48, wherein the aggregated data form further comprises an unstructured file indicating how the plurality of medical items should be laid out in preparation for the surgeon to perform the surgery type at the facility.

Example 50 is a method as in any of Examples 1-17 or Examples 32-49, wherein the aggregated data form further comprises an indication that at least one of the plurality of medical items is a special handling item that is not included in the inventory management solution for the facility, wherein the special handling item is associated with contact information for acquiring the special handling item in preparation for the surgeon to perform the surgery type at the facility.

Example 51 is a method as in any of Examples 1-17 or Examples 32-50, further comprising rendering a user interface accessible to a user, wherein the user interface provides a means for a user to check off each of the plurality of medical items in preparation for the surgeon to perform the surgery type at the facility.

Computer storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium, which can be used to store desired program code means in the form of computer-executable instructions or data structures and which can be accessed by a general purpose or special purpose computer.

Those skilled in the art will appreciate that the disclosure may be practiced in network computing environments with many types of computer system configurations, including, an in-dash vehicle computer, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, various storage devices, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be in both local and remote memory storage devices.

Further, although specific implementations of the disclosure have been described and illustrated, the disclosure is not to be limited to the specific forms or arrangements of parts so described and illustrated. The scope of the disclosure is to be defined by the claims appended hereto, any future claims submitted here and in different applications, and their equivalents.

What is claimed is:

1. A method comprising:
generating an aggregated data form comprising surgical preference data, wherein the aggregated data form identifies a plurality of medical items, and wherein the aggregated data form is associated with a surgeon, a facility, and a surgery type;
electronically communicating with an inventory management solution associated with the facility to retrieve inventory data for the plurality of medical items;
providing the aggregated data form and the inventory data to a machine learning algorithm;
receiving from the machine learning algorithm an amendment suggestion for the aggregated data form, wherein the amendment suggestion comprises one or more of:
an amendment to a quantity of a first medical item of the plurality of medical items;
an identity of a first product satisfying the first medical item; or
an addition of a second medical item not included in the plurality of medical items of the aggregated data form;
wherein the machine learning algorithm is trained on the inventory data and further on a plurality of aggregated data forms associated with one or more of the same surgeon, the same facility, or the same surgery type.

2. The method of claim 1, further comprising:
ingesting an unstructured file comprising text;
providing the unstructured file to a file analysis machine learning algorithm configured to execute optical character recognition processing to identify textual characters in the unstructured file, wherein the file analysis machine learning algorithm identifies the textual characters by identifying patterns of light portions and dark portions in the unstructured file;
receiving an output comprising the textual characters identified by the file analysis machine learning algorithm;
processing the textual characters to identify a plurality of names for the plurality of medical items;
assigning the plurality of medical items to the aggregated data form, and storing the aggregated data form on a cloud-based database; and
generating a summary file comprising information from the aggregated data form, wherein the summary file comprises structured data and unstructured data.

3. The method of claim 1, wherein the inventory data comprises surgical usage data for the facility, and wherein the surgical usage data comprises an indication of which products were utilized in a plurality of surgical procedures performed at the facility.

4. The method of claim 3, wherein the machine learning algorithm is trained to assess the surgical usage data for the facility;
wherein the amendment suggestion output by the machine learning algorithm comprises the amendment to the quantity of the first medical item; and
wherein the machine learning algorithm suggests the amendment to the quantity based upon one or more of:
determining the same surgeon historically utilizes more of the first medical item or fewer of the first medical item when performing the surgery type at the facility, as determined based upon the surgical usage data;
determining that any surgeon at the facility historically utilizes more of the first medical item or fewer of the first medical item when performing the surgery type at the facility, as determined based upon the surgical usage data; or
determining that any of a plurality of surgeons request more of the first medical item or fewer of the first medical item when performing the surgery type, as determined based upon a plurality of aggregated data forms associated with the plurality of surgeons.

5. The method of claim 1, wherein the machine learning algorithm suggests the first product satisfying the first medical item based upon one or more of:
determining the first product is available at the facility, as determined based upon the inventory data;
determining the first product is a most affordable product satisfying the first medical item available at the facility, as determined based upon the inventory data; or
determining the first product satisfies the first medical item, as determined based upon medical device data ingested from a medical device database.

6. The method of claim 1, wherein the machine learning algorithm suggests the first product satisfying the first medical item based upon determining the first product is utilized to satisfy the first medical item by one or more other surgeons performing the same surgery type, as determined based upon a plurality of aggregated data forms associated with the plurality of surgeons.

7. The method of claim 1, wherein the machine learning algorithm suggests the addition of the second medical item based upon determining the second medical item is utilized to perform the same surgery type by one or more of a plurality of other surgeons, as determined based upon a plurality of aggregated data forms associated with the plurality of other surgeons.

8. The method of claim 1, further comprising:
ingesting medical device data from a medical device database, wherein the medical device data comprises a listing of a plurality of medical devices approved for use at the facility;
ingesting pharmaceutical data from a pharmaceutical database, wherein the pharmaceutical data comprises a listing of a plurality of pharmaceuticals approved for use at the facility;
matching each of the plurality of medical items identified on the aggregated data from with at least one medical device of the plurality of medical devices, or at least one pharmaceutical of the plurality of pharmaceuticals.

9. The method of claim 1, further comprising:
storing data for a plurality of medical devices on a database, wherein the data includes a barcode associated with each of the plurality of medical devices;
determining that a first medical device of the plurality of medical devices satisfies the first medical item identified in the aggregated data form;
receiving an indication that a barcode scanner scanned a first barcode associated with the first medical device in preparation for performing the surgery type;
matching the first barcode with the first medical device;
matching the first medical device with the aggregated data form; and
marking the first medical item on the aggregated data form as having been collected in preparation for performing the surgery type.

10. The method of claim 1, further comprising generating a scannable code associated with the surgical preference data, wherein the scannable code redirects to one or more of:
a complete version of the aggregated data form comprising all information associated with the aggregated data form; or
a cleaned version of the aggregated data form that does not comprise personal health information.

11. The method of claim 1, further comprising:
storing a plurality of aggregated data forms on a database, wherein each of the plurality of aggregated data forms is associated with the facility; and
exporting each of the plurality of aggregated data forms to a compressed file to be stored on local memory at the facility;
wherein the compressed file is accessible at the facility in the event of a network outage.

12. The method of claim 1, further comprising communicating with a web scraping module, wherein the web scraping module is configured to extract structured data from one or more web pages, wherein the structured data comprises information regarding one or more medical products, the information comprising one or more of a name, unique product code, regulatory approval status, current price, current availability, or safety rating.

13. The method of claim 1, wherein the aggregated data form comprises a plurality of data buckets, and wherein one or more of the plurality of data buckets is a required data bucket that must be filled with data for the aggregated data form to be complete, and wherein the method further comprises:
determining whether the aggregated data form is complete based on whether each of the required data buckets is filled; and
in response to determining that each of the required data buckets is filled, generating a message querying a user whether the aggregated data bucket is complete.

14. The method of claim 1, further comprising:
storing a plurality of aggregated data forms on a cloud-based database, wherein the plurality of aggregated data forms is associated with the same facility and the same surgery type; and
rendering a user interface accessible to a user when the user is preparing or viewing the aggregated data form, wherein the user interface comprises, for each of the plurality of medical items on the aggregated data form, an indication of a proportion of the plurality of aggregated data forms that includes the corresponding medical item.

15. The method of claim 1, further comprising:
storing a plurality of aggregated data forms on a cloud-based database, wherein the plurality of aggregated data forms is associated with the same facility and the same surgery type; and
rendering a user interface accessible to a user when the user is preparing or viewing the aggregated data form, wherein the user interface comprises a suggestion of additional medical items to be included in the aggregated data form;
wherein the additional medical items are included in at least a portion of the plurality of aggregated data form.

16. The method of claim 1, wherein at least one of the plurality of medical items is a special handling item, and wherein the special handling item is not included in the inventory management solution associated with the facility.

17. The method of claim 1, wherein the plurality of medical items comprises one or more of medical devices or pharmaceuticals to be present when the surgeon performs the surgery type at the facility.

18. The method of claim 1, wherein the aggregated data form further comprises an unstructured file indicating how the plurality of medical items should be laid out in preparation for the surgeon to perform the surgery type at the facility.

19. The method of claim 1, wherein the aggregated data form further comprises an indication that at least one of the plurality of medical items is a special handling item that is not included in the inventory management solution for the facility, wherein the special handling item is associated with contact information for acquiring the special handling item in preparation for the surgeon to perform the surgery type at the facility.

20. The method of claim 1, further comprising rendering a user interface accessible to a user, wherein the user interface provides a means for a user to check off each of the plurality of medical items in preparation for the surgeon to perform the surgery type at the facility.
